SEQUENCE LISTING (28 sequences)

SEQ ID NO: 1 - protein, 21 aa, homo sapiens
  CD8alpha signal peptide
MALPVTALLLPLALLLHAARP

SEQ ID NO: 2 - protein, 20 aa, homo sapiens
  signal peptide
METDTLLLWVLLLWVPGSTG

SEQ ID NO: 3 - protein, 16 aa, homo sapiens
  FcgRIIIa hinge
GLAVSTISSFFPPGYQ

SEQ ID NO: 4 - protein, 45 aa, homo sapiens
  CD8alpha hinge
TTTPAPRPPTPAPTIASQPLSLRPEACRPAAGGAVHTRGLDFACD

SEQ ID NO: 5 - protein, 231 aa, homo sapiens
  IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, homo sapiens
  CD8alpha transmembrane domain
IYIWAPLAGTCGVLLLSLVITLYC

SEQ ID NO: 7 - protein, 27 aa, homo sapiens
  41BB transmembrane domain
IISFFLALTSTALLFLLFFLTLRFSVV

SEQ ID NO: 8 - protein, 42 aa, homo sapiens
  Fragment of 4-1BB (residues 214-255)
KRGRKKLLYIFKQPFMRPVQTTQEEDGCSCRFPEEEEGGCEL

SEQ ID NO: 9 - protein, 112 aa, homo sapiens
  fragment of T-cell surface glycoprotein CD3 zeta chain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, artificial sequence
  G4Sx3 linker sequence
GGGGSGGGGSGGGGS

SEQ ID NO: 11 - protein, 116 aa, artificial sequence
  139- heavy chain
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSS

SEQ ID NO: 12 - protein, 107 aa, artificial sequence
  139- light chain variable region
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIK

SEQ ID NO: 13 - protein, 118 aa, artificial sequence
  MR1- heavy chain
QVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTV

SEQ ID NO: 14 - protein, 108 aa, artificial sequence
  MR1- light chain
DIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKAL

SEQ ID NO: 15 - protein, 461 aa, artificial sequence
  139-v1 polypolypeptide CAR sequence
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSGGGGSGGGGSGGGGSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 647 aa, artificial sequence
  139-v2 polypolypeptide CAR sequence
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSGGGGSGGGGSGGGGSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 464 aa, artificial sequence
  MR1-v1 polypolypeptide CAR sequence
QVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVGGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 650 aa, artificial sequence
  MR1-v2 polypolypeptide CAR sequence
QVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVGGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 49 bp, homo sapiens
  Target TALEN TRAC_T01
ttgtcccacagatatccagaaccctgaccctgccgtgtaccagctgaga

SEQ ID NO: 20 - protein, 530 aa, artificial sequence
  TAL binding domain  TRAC_T01-L
LTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALE

SEQ ID NO: 21 - protein, 530 aa, artificial sequence
  TAL binding domain TRAC_T01-R
LTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALE

SEQ ID NO: 22 - DNA, 2814 bp, artificial sequence
  polynucleotide encoding  TRAC_T01-L TALEN
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 23 - DNA, 2832 bp, artificial sequence
  polynucleotide encoding TRAC_T01-R TALEN
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 24 - protein, 482 aa, artificial sequence
  139-v3 polypolypeptide CAR sequence
MALPVTALLLPLALLLHAARPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKGGGGSGGGGSGGGGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 672 aa, artificial sequence [sequence omitted]

SEQ ID NO: 26 - protein, 485 aa, artificial sequence
  MR1-v3 polypolypeptide CAR sequence
MALPVTALLLPLALLLHAARPQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVGGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 675 aa, artificial sequence [sequence omitted]

SEQ ID NO: 28 - protein, 548 aa, artificial sequence
  Rosenberg CAR
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2017/0275366 A1
(43) Pub. Date: Sep. 28, 2017

(54) EGFRvIII Specific Chimeric Antigen Receptor For Cancer Immunotherapy

(71) Applicant: PFIZER INC., New York, NY (US)

(72) Inventor: Cecile SCHIFFER-MANNIOUI, Villiers-sur-Marne (FR)

(21) Appl. No.: 15/526,649

(22) PCT Filed: Jul. 29, 2015
(86) PCT No.: PCT/EP2015/067439

§ 371 Date: May 12, 2017

(30) Foreign Application Priority Data
Jul. 29, 2014 (DK) PA201470466

(51) Int. Cl. C07K 16/28; C07K 14/705; A61K 35/17; C07K 14/725

(52) U.S. Cl. CPC C07K 16/2863; C07K 14/7051; C07K 14/70578; C07K 14/70517; A61K 35/17; C07K 2317/56; C07K 2317/53; C07K 2319/03; C07K 2317/24; C07K 2317/622

(57) ABSTRACT

The present invention relates to Chimeric Antigen Receptors (CAR) that are recombinant chimeric proteins able to redirect immune cell specificity and reactivity toward selected membrane antigens, and more particularly in which extracellular ligand binding is a scFV derived from an EGFRvIII monoclonal antibody, conferring specific immunity against EGFRvIII positive cells. The TCR KO engineered immune cells endowed with such CARs are particularly suited for treating lung cancer, anal cancers and glioblastoma multiforme.

### FIELD OF THE INVENTION

The present invention relates to Chimeric Antigen Receptors (CAR) that are recombinant chimeric proteins able to redirect immune cell specificity and reactivity toward EGFRvIII, a cell surface glycoprotein found on human tumors including glioblastomas, gliomas, non-small-cell lung carcinomas, ovarian carcinomas and prostate carcinomas. The CARs according to the invention are particularly useful to treat malignant cells bearing EGFRvIII antigen, when expressed in T-cells or NK cells. The resulting engineered immune cells display high level of specificity toward malignant cells, conferring safety and efficiency for immunotherapy.

### BACKGROUND OF THE INVENTION

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T-cell cytotoxicity. However, they failed to provide prolonged expansion and anti-tumor activity in vivo. Signaling domains from co-stimulatory molecules, as well as transmembrane and hinge domains have been added to form CARs of second and third generations, leading to some successful therapeutic trials in humans, where T-cells could be redirected against malignant cells expressing CD19 (June et al., 2011). However, the particular combination of signaling domains, transmembrane and co-stimulatory domains used with respect to CD19 ScFv, was rather antigen-specific and cannot be expanded to any antigen markers.

Malignant gliomas are the most common and deadly brain tumors. Nevertheless, survival for patients with glioblastoma, the most aggressive glioma, although individually variable, has improved from an average of 10 months to 14 months after diagnosis in the last 5 years due to improvements in the standard of care. Radiotherapy has been of key importance to the treatment of these lesions for decades, and the ability to focus the beam and tailor it to the irregular contours of brain tumors and minimize the dose to nearby critical structures with intensity-modulated or image-guided techniques has improved greatly. Temozolomide, an alkylating agent with simple oral administration and a favorable toxicity profile, is used in conjunction with and after radiotherapy. Newer surgical techniques, such as fluorescence-guided resection and neuroendoscopic approaches, have become important in the management of malignant gliomas (Van Meir et al, 2010).

In spite of these advances, there is still a great need of non-invasive therapies for glioblastoma. In particular there is a need for “off the shelve CAR T cells” for their use in the treatment of EGFRvIII-mediated pathologies, in particular for the treatment of lung cancer, anal cancer residual or recurrent EGFRvIII+ Glioma and glioblastoma multiforme (GBM), preferably residual or recurrent EGFRvIII+ Glioma or GBM. Here, the inventors have developed an effective chimeric antigen receptor targeting the Epidermal growth factor receptor variant III (EGFRvIII) as an antigen, which is a glycoprotein uniquely expressed in glioblastoma, but not in normal brain tissues, referred to as P00533 in the Uniprot database (encoded by the gene having the NCBI reference NM-00522). This invention opens the way for treating human tumors such as glioblastoma by immunotherapy, especially using CAR-expressing T cells, with significant clinical advantage.

### SUMMARY OF THE INVENTION

The present invention provides the following objects that solve the problems herein identified:
1. An EGFRvIII specific chimeric antigen receptor (EGFRvIII CAR) having one of the polypeptide structure selected from V1 to V6 as illustrated in FIG. 2, said structure comprising:
   an extra cellular ligand binding-domain comprising a VH and a VL from a monoclonal anti-EGFRvIII antibody, optionally a linker, in particular a linker of formula (G4S)n wherein n is 1-3, preferably n=3 (of SEQ ID NO. 10.),
      a hinge,
      a transmembrane domain and
      a cytoplasmic domain including a CD3 zeta signaling domain and a co-stimulatory domain from 4-1BB.
2. The present invention provides an EGFRvIII specific CAR according to 1 comprising:
   an extracellular ligand binding-domain comprising a VH and a VL from a monoclonal anti-EGFRvIII antibody, a linker, of formula (G4S)3 (of SEQ ID NO. 10.),
      a hinge,
      a transmembrane domain from CD8 alpha and
      a cytoplasmic domain including a CD3 zeta signaling domain and a co-stimulatory domain from 4-1 BB.
3. The present invention provides an EGFRvIII specific CAR according to 1 or 2 comprising no domain from human CD28, in particular no co-stimulatory domain from human CD28.
4. The present invention provides an EGFRvIII specific CAR according to any one of 1 to 3, wherein said VH and VL have at least 80% identity with a polypeptide sequence selected from SEQ ID NO. 11 to 14, optionally humanized.
5. The present invention provides an EGFRvIII specific CAR according to any one of 1 to 4, wherein said co-stimulatory domain from 4-1 BB has at least 80% identity with SEQ ID NO.8, optionally humanized.
6. The present invention provides an EGFRvIII specific CAR according to any one of 1 to 5, wherein said CD3 zeta signaling domain has at least 80% identity with SEQ ID NO. 9, optionally humanized.
7. The present invention provides an EGFRvIII specific CAR according to any one of 1 to 6, wherein said CD8α transmembrane domain has at least 80% identity with SEQ ID NO.6, optionally humanized.
8. The present invention provides an EGFRvIII specific CAR according to any one of 1 to 7, further comprising another extracellular ligand binding domain which is not specific for EGFRvIII.
9. The present invention provides an EGFRvIII specific CAR according to any one of 1 to 8, further comprising a signal peptide.
10. The present invention provides an EGFRvIII specific CAR according to 9, wherein said signal peptide has at least 80% sequence identity with SEQ ID NO.1 or SEQ ID NO.2, optionally humanized.
11. The present invention provides an EGFRvIII specific CAR according to any one of 1 to 10, wherein said structure V1 comprises a FcγRIIIα hinge and CD8α transmembrane domain.
12. The present invention provides an EGFRvIII specific CAR according to 11, wherein said FcγRIIIα hinge has at least 80% identity with SEQ ID NO.3, optionally humanized.
13. The present invention provides an EGFRvIII specific CAR according to any one of 1 to 10, wherein said structure V3 comprises a CD8α hinge and a CD8α transmembrane domain.
14. The present invention provides an EGFRvIII specific CAR according to 13, wherein said CD8α hinge has at least 80% identity with SEQ ID NO.4, optionally humanized.
15. The present invention provides an EGFRvIII specific CAR according to any one of 1 to 10, wherein said structure V5 comprises an IgG1 hinge and a CD8α transmembrane domain.
16. The present invention provides an EGFRvIII specific CAR according to 15, wherein said IgG1 hinge has at least 80% identity with SEQ ID NO.5, optionally humanized.
17. The present invention provides an EGFRvIII specific CAR of structure V1 according to any one of 1-10, or 11-12 which comprises a polypeptide sequence having at least 80% identity with SEQ ID NO. 15 or SEQ ID NO.17.
18. The present invention provides advantageously, an EGFRvIII specific CAR of structure V3 according to any one of 1-10 or 13-14 having at least 80% identity with a sequence selected from SEQ ID NO. 24 and SEQ ID NO. 26.
19. The present invention provides an EGFRvIII specific CAR of structure V5 according to any one of 1-10 or 15-16 having at least 80% identity with a sequence selected from SEQ ID NO.25, and SEQ ID NO.27.
20. The present invention provides an EGFRvIII specific CAR according to any one of 1-10 or 13-14 or 18 having a signal peptide, a hinge and a TM-domain from CD8α.
In one embodiment, said EGFRvIII CAR of the invention as described from 1 above to 20 below, allows the binding of T cells expressing said EGFRvIII CAR, preferably the binding of engineered T cell as below expressing said EGRFvIII CAR, to EGFRvIII, preferably to EGFRvIII-expressing cells, more preferably to EGFRvIII-expressing cancer cells.
In another embodiment, said EGFRvIII CAR of the invention as described from 1 above to 20 below, allows the binding of T cells expressing said EGFRvIII CAR, preferably the binding of engineered T cell as below expressing said EGRFvIII CAR, to EGFRvIII, preferably to EGFRvIII-expressing cells, more preferably to EGFRvIII-expressing cancer cells and destroys said EGFRvIII-expressing cells, more preferably to EGFRvIII-expressing cancer cells.
21. The present invention provides a polynucleotide encoding an EGFRvIII specific CAR according to any one of 1 to 20.
The present invention provides an EGFRvIII specific CAR according to any one of 1 to 20 wherein the polypeptide sequence contains no sequence from human CD28.
In particular embodiments, the EGFRVIII CAR of the invention from 1 to 20 does not include a sequence having at least 1, at least 2, at least 3, at least 4, at least 5, at least 6, at least 7 at least 8, at least 9 at least 10 amino acids identity with human CD28.
In particular embodiments, the intracytoplasmic and/or transmembrane domain of the EGFRVIII CAR of the invention from 1 to 20 does not include human CD28-derived sequence, in particular has no sequence having at least 1, at least 2 at least 3, at least 4 at least 5, at least 6, at least 7 at least 8, at least 9 at least 10 amino acids identity with human CD28.
22. The present invention provides an expression vector comprising a polynucleotide of 21.
23. The present invention provides an expression vector according to 22 wherein said vector is a lentiviral vector, preferably a lentiviral vector pCLD27600.
In particular embodiments, said expression vector allows the stable expression of the EGFRvIII CAR of the invention as described from 1 above to 20.
24. The present invention provides an engineered immune cell expressing at the cell surface membrane an EGFRvIII specific CAR according to any one of 1 to 20.
25. The present invention provides an engineered immune cell according to 24, derived from an immune cell selected from inflammatory T-lymphocytes, cytotoxic T-lymphocytes, regulatory T-lymphocytes or helper T-lymphocytes, preferably from cytotoxic T-lymphocytes.
The present invention provides an engineered immune cell according to 24, derived from cytotoxic T-lymphocytes.
26. The present invention provides an engineered immune cell according to 24, derived from a NK cell.
27. The present invention provides an engineered cell according to any one of 24 to 26, wherein expression of TCR is suppressed.
28. The present invention provides an engineered cell according to any one of 24 to 27, wherein expression of at least one MHC protein, preferably β2m or HLA, is suppressed.
29. The present invention provides an engineered cell according to any one of 24 to 28, wherein said cell is resistant to at least one immune suppressive or chemotherapy drug.
30. The present invention provides an engineered cell according to any one of 24 to 29 for use in therapy to prevent or treat a condition in a patient.
31. The present invention provides an engineered cell for use in therapy according to 30 for the treatment of a pre-malignant or malignant cancer condition characterized by EGFRvIII-expressing cancer cells.
32. The present invention provides an engineered cell for use in therapy according to any one of 30 to 31 for the treatment of a condition characterized by an overabundance of EGFRvIII-expressing cancer cells.
33. The present invention provides an engineered cell for use in therapy according to any one of 30 to 32 for use in therapy, wherein the condition is a cancer selected from lung cancer, anal cancer residual or recurrent EGFRvIII+ Glioma and glioblastoma multiforme (GBM), preferably residual or recurrent EGFRvIII+ Glioma or GBM.
The present invention provides an engineered cell for use in therapy according to any one of 30 to 32 for use in therapy, wherein the condition is GBM.
The present invention provides an engineered cell for use in therapy according to any one of 30 to 32 for use in therapy, wherein the condition is recurrent EGFRvIII+ Glioma.
Also disclosed herein are engineered cell of the invention from 24 to 32 wherein said EGFRvIII CAR of the invention binds to EGFRvIII, preferably to EGFRvIII-expressing cells, more preferably to EGFRvIII-expressing cancer cells.
Preferably, engineered cell of the invention as disclosed according to any of the embodiments 24 to 32, binds to EGFRvIII-expressing cancer cells and affect said survival of EGFRvIII-expressing cancer cells, more preferably engineered cell of the invention as disclosed here according to any of the embodiments below, improve the survival of patients suffering glioma, in particular GBM.
34. The present invention provides a method of impairing a cancer cell comprising contacting said cancer cell with an engineered cell according to any one of 24 to 29 in an amount effective to cause impairment of said cancer cell.
35. The present invention provides a method of engineering an immune cell comprising:
   (a) Providing an immune cell,
   (b) Expressing at the surface of said cell at least one EGFRvIII specific CAR according to any one of 1 to 20.
36. The present invention provides a method of engineering an immune cell of 35 comprising:
   (a) Providing an immune cell,
   (b) Introducing into said cell at least one polynucleotide encoding said EGFRvIII specific CAR, according to 21,
   (c) Expressing said polynucleotide into said cell.
37. The present invention provides a method of engineering an immune cell according to any one of 35-36 comprising:
   (a) Providing an immune cell,
   (b) Introducing into said cell at least one polynucleotide encoding said EGFRvIII specific CAR,
   (c) Introducing at least one other CAR which is not specific for EGFRvIII.
38. The present invention provides a method of treating a subject in need thereof comprising:
   (a) Providing an engineered cell according to any one of 24 to 29 expressing at the surface an EGFRvIII specific CAR;
   (b) Administrating said engineered cells to said patient.
39. The present invention provides a method according to 38, wherein said engineered cell is prepared using an immune cell provided by a donor.
40. The present invention provides a method according to 39, wherein said donor is a patient, preferably said patient will be treated using its own immune cells engineered according to any one of 35 to 37.

### The Present Invention Also Provides:

1. A chimeric antigen receptor (CAR) comprising an antigen binding domain of an antibody specific for EGFRVIII, (EGFRVIII CAR) comprising an antigen binding domain of an antibody specific for EGFRVIII, a leader sequence, an extracellular hinge domain, a transmembrane domain, and an intracellular T cell signaling domain.

The EGFRVIII CAR according to 1, wherein the antigen binding domain comprises a light chain variable region comprising SEQ ID NO: 11 or 13.

The EGFRVIII CAR according to 1 or 2, wherein the antigen binding domain comprises a heavy chain variable region comprising SEQ ID NO: 12 or 14.

4. The EGFRVIII CAR according to any one of 1-3, wherein the antigen binding domain comprises a linker peptide comprising SEQ ID NO: 10.

5. The EGFRVIII CAR according to any one of 1-4, wherein the antigen binding domain comprises a leader sequence comprising SEQ ID NO: 1 or 2.

6. The EGFRVIII CAR according to any one of 1-5, wherein the antigen binding domain comprises a leader sequence of SEQ ID NO: 1.

7. The EGFRVIII CAR according to any one of 1-6, further comprising an extracellular hinge domain.

8. The EGFRVIII CAR according to any one of 1-7, wherein the leader sequence, extracellular hinge domain and transmembrane domain comprise a sequence from CD8 alpha chain (SEQ ID NO: 6).

9. The EGFRVIII CAR according to any of 1-8, wherein the intracellular T cell signaling domain comprises, 4-1BB SEQ ID NO; 8, and CD3ζ (SEQ ID NO; 9), preferably no CD28 sequence.

10. A nucleic acid comprising a nucleotide sequence encoding the EGFRVIII CAR according to any of 1-9.

11. A recombinant expression vector comprising the nucleic acid of 10.

12. An isolated primary cell comprising the recombinant expression vector of 11.

13. A TCR-KO isolated, primary cell comprising the recombinant expression vector of 11

14. A TCR-KO isolated, primary cell comprising the EGFRVIII CAR of 1 to 9

15. A population of primary cells comprising at least one isolated primary cell of 12, 13 or 14.

16. A pharmaceutical composition comprising the EGFRVIII CAR of 1-9, the nucleic acid of 10, the recombinant expression vector of 11, the isolated primary cell of 12, 13 or 14, the population of primary cells of 15, and a pharmaceutically acceptable carrier.

17. The EGFRVIII CAR of 1-9, the nucleic acid of 10, the recombinant expression vector of 11, the isolated primary cell of 12, 13 or 14, the population of isolated primary cells of 15 or the pharmaceutical composition of 16 for use in the treatment or prevention of cancer in a host, preferably suffering glioma, more preferably a glioblastoma.
1. The present invention finally provides an EGFRvIII specific chimeric antigen receptor (CAR) having one of the polypeptide structure selected from V1 to V4 as illustrated in FIG. 2, said structure comprising an extra cellular ligand binding-domain comprising VH and VL from a monoclonal anti-EGFRvIII antibody, a hinge, a transmembrane domain and a cytoplasmic domain including a CD3 zeta signaling domain and a co-stimulatory domain from 4-1 BB.
2. A EGFRvIII specific CAR according to 1, wherein said structure V1 comprises a FcγRIIIα hinge and CD8α transmembrane domain.
3. A EGFRvIII specific CAR according to 1, wherein said structure V2 comprises a FcγRIIIα hinge and a 4-1BB transmembrane domain.
4. A EGFRvIII specific CAR according to 1, wherein said structure V3 comprises a CD8α hinge and a CD8α transmembrane domain.
5. A EGFRvIII specific CAR according to 1, wherein said structure V4 comprises a CD8α hinge and a 4-1 BB transmembrane domain.
6. A EGFRvIII specific CAR according to any one of 1 to 5, wherein said VH and VL have at least 80% identity with a polypeptide sequence selected from SEQ ID NO. 11 to 14.
7. A EGFRvIII specific CAR according to any one of 1 to 6, wherein co-stimulatory domain from 4-1BB has at least 80% identity with SEQ ID NO.8.
8. A EGFRvIII specific CAR according to any one of 1 to 6, wherein said CD3 zeta signaling domain has at least 80% identity with SEQ ID NO. 9.
9. A EGFRvIII specific CAR according to any one of 1 or 2, wherein said FcγRIIIα hinge has at least 80% identity with SEQ ID NO.3.
10. A EGFRvIII specific CAR according to any one of 3 or 4, wherein said CD8α hinge has at least 80% identity with SEQ ID NO.4.
11. A EGFRvIII specific CAR according to any one of 5, wherein said IgG1 hinge has at least 80% identity with SEQ ID NO.5.
12. A EGFRvIII specific CAR according to any one of 2 or 4, wherein said CD8α transmembrane domain has at least 80% identity with SEQ ID NO.6.
13. A EGFRvIII specific CAR according to any one of 1, 3 or 5, wherein said 4-1BB transmembrane domain has at least 80% identity with SEQ ID NO.7.
14. A EGFRvIII specific CAR according to any one of 1 to 13 further comprising another extracellular ligand binding domain which is not specific for EGFRvIII.
15. A EGFRvIII specific CAR of structure V1 according to 2, which comprises a polypeptide sequence having at least 80% identity with SEQ ID NO. 15 and SEQ ID NO.17.
16. A EGFRvIII specific CAR of structure V2 according to 3, which comprises a polypeptide sequence having at least 80% identity with SEQ ID NO. 16 and SEQ ID NO.18.
17. A EGFRvIII specific CAR according to any one of 1 to 16, further comprising a signal peptide.
18. A EGFRvIII specific CAR according to 17, wherein said signal peptide has at least 80% sequence identity with SEQ ID NO.1 or SEQ ID NO.2.
19. A polynucleotide encoding a chimeric antigen receptor according to any one of 1 to 18.
20. An expression vector comprising a nucleic acid of 6 or 7.
21. An engineered immune cell expressing at the cell surface membrane an EGFRvIII specific chimeric antigen receptor according to any one of 1 to 18.
22. An engineered immune cell according to 21, derived from inflammatory T-lymphocytes, cytotoxic T-lymphocytes, regulatory T-lymphocytes or helper T-lymphocytes.
23. An engineered immune cell according to 21, wherein it is derived from a NK cell.
24. An engineered cell according to any one of 21 to 23 for use in therapy.
25. An engineered cell according to any one of 21 to 23 for use in human therapy.
26. An engineered cell according to any one of 21 to 25 for use in therapy, wherein the condition is a pre-malignant or malignant cancer condition characterized by EGFRvIII-expressing cells.
27. An engineered cell according to any one of 21 to 26 for use in therapy, wherein the condition is a condition which is characterized by an overabundance of EGFRvIII-expressing cells.
28. An engineered cell according to any one of 21 to 27 for use in therapy, wherein the condition is a cancer condition.
29. An engineered cell according to any one of 21 to 28 for use in therapy, wherein the cancer condition is lung cancer, anal cancers or glioblastoma multiforme.
30. An engineered cell according to any one of 21 to 29, wherein expression of TCR is suppressed in said immune cell.
31. An engineered cell according to any one of 21 to 30, wherein expression of at least one MHC protein, preferably β2m or HLA, is suppressed in said immune cell.
32. An engineered cell according to any one of 21 to 31, wherein said cell is mutated to confer resistance to at least one immune suppressive or chemotherapy drug.
33. A method of impairing a cancer cell comprising contacting said cell with an engineered cell according to any one of 21 to 32 in an amount effective to cause impairment of said cancer cell.
34. A method of engineering an immune cell comprising:
   (a) Providing an immune cell,
   (b) Expressing at the surface of said cell at least one EGFRvIII specific chimeric antigen receptor according to any one of 1 to 19.
35. The method of engineering an immune cell of 34 comprising:
   (a) Providing an immune cell,
   (b) Introducing into said cell at least one polynucleotide encoding said EGFRvIII specific chimeric antigen receptor,
   (c) Expressing said polynucleotide into said cell.
36. The method of engineering an immune cell of 34 comprising:
   (a) Providing an immune cell,
   (b) Introducing into said cell at least one polynucleotide encoding said EGFRvIII specific chimeric antigen receptor,
   (c) Introducing at least one other chimeric antigen receptor which is not specific for EGFRvIII.
37. A method of treating a subject in need thereof comprising:
   (a) Providing a immune cell expressing at the surface a EGFRvIII specific Chimeric Antigen Receptor according to any one of 1 to 19;
   (b) Administrating said immune cells to said patient.
38. A method according to 37, wherein said immune cell is provided from a donor.
39. A method according to 37, wherein said immune cell is provided from the patient himself.

The inventors have generated an EGFRvIII specific CAR having different structure and comprising different scFV derived from different EGFRvIII specific antibodies. Preferred CAR polypeptides of the invention comprise an amino acid sequence selected from SEQ ID NO.15 to 19.

More preferred CARs of the invention are EGFRvIII specific CAR with a V3 or a V5 architecture (Table A and B), even more preferred of V3 architecture (Table A) and even more preferably CARs of the invention are EGFRvIII specific CAR having an amino acid sequence selected from SEQ ID NO, 24 and SEQ ID NO, 26, optionally humanized.

Even more preferred CARs of the invention are humanized CARs selected from SEQ ID NO, 24, SEQ ID NO, 25, SEQ ID NO, 26 and SEQ ID NO, 27 wherein at least 1, at least 2, at least 3, at least 5, at least 8, at least 10 amino acids has been changed to reduce the HAMA response while keeping a selectivity and affinity for human EGFRvIII similar or better to that the non-humanized EGFRvIII CAR.

Term “similar” means having the affinity of the non-humanized CAR with a standard deviation of from 0.05 to 0.5 (n=2). Term “improved” means having the affinity of the non-humanized EGFRvIII CAR increased by a factor of at least 1.2.

According to the present invention, humanized also means an EGFRvIII CAR having at least 80% identity with the wt EGFRvIII CAR or original EGFRvIII CAR sequence.

Following non-specific activation in vitro (e.g. with anti CD3/CD28 coated beads and recombinant IL2), T-cells from donors have been transformed with polynucleotides expressing these CARs using viral transduction. In certain instances, the T-cells were further engineered to create non-alloreactive T-cells, more especially by disruption of a component of TCR (αβ-T-Cell receptors) to prevent Graft versus host reaction.

The resulting engineered T-cells displayed reactivity in-vitro against EGFRvIII positive cells to various extend, showing that the CARs of the present invention contribute to antigen dependent activation, and also proliferation, of the T-cells, making them useful for immunotherapy.

The resulting engineered T-cells displayed reactivity in-vivo against EGFRvIII positive cells, showing that the CARs of the present invention contribute to antigen dependent activation, and also proliferation, of the T-cells in vivo, making them useful for immunotherapy.

The polypeptides and polynucleotide sequences encoding the CARs of the present invention are detailed in the present specification.

The engineered immune cells of the present invention are particularly useful for therapeutic applications, such as for treating multiple myeloma.

The engineered immune cells of the present invention are particularly useful for therapeutic applications, such as for treating glioblastoma multiforme (GBM) (also known as glioblastoma, astrocytoma grade IV, and grade IV astrocytoma). Preferably, the cancer is characterized by cells expressing EGFRvIII.

Also, disclosed herein are engineered immune cells endowed with EGFRvIII CAR constructs of the invention, preferably an EGFRvIII CAR with a V3 architecture, wherein the hinge domain is a hinge domain from CD8alpha.

An engineered immune cell of the invention may be endowed with EGFRvIII CAR constructs combining a hinge from CD8 with a signal peptide and or a transmembrane region also from CD8alpha.

### BRIEF DESCRIPTION OF THE FIGURES

FIG. 1: Schematic representation of an engineered immune cell according to the invention. The engineered immune cell presented in this figure is a T-cell transduced with a retroviral polypeptide encoding CAR. This T-cell is further engineered to allow a better and safer engraftment into the patient, which is optional within the frame of the present invention. X gene may be for instance a gene expressing a component of TCR (TCRalpha or TCRbeta), Y may be a gene involved into the sensitivity of T-cells to immune-suppressive drugs like CD52 (with respect to Campath) or HPRT (with respect to 6-Thioguanine).

FIG. 2: Schematic representation of the different CAR Architecture (V1 to V4) and V5 to V6.

FIG. 3: Schematic representation of EGFRvIII CAR constructs.

FIG. 4: Backbone for CAR mRNA production.

FIG. 5: Backbone for CAR lentiviral vector production.

FIG. 6: EGFRvIII CAR expression in primary T cells analyzed by FACS

FIG. 7: U87 glioma cells overexpressing EGFRVI or EGFRVIII proteins characterization by Western-Blot.

FIG. 8: EGFRvIII CAR T degranulation capacity assessed by FACS analysis after coculture with target cells.

FIG. 9: Cytotoxicity assay of EGFRvIII CART cells of the invention.

TABLE 1
Sequence of the different EGFRvIII
CAR components of the invention

| Functional | | Raw amino |
|---|---|---|
| domains | SEQ ID # | acid sequence |
| CD8α signal | SEQ ID NO. 1 | MALPVTALLLPLA |
| peptide | | LLLHAARP |
| Alternative | SEQ ID NO. 2 | METDTLLLWVLLL |
| signal peptide | | WVPGSTG |
| FcγRIIIα hinge | SEQ ID NO. 3 | GLAVSTISSFFPP |
| | | GYQ |
| CD8α hinge | SEQ ID NO. 4 | TTTPAPRPPTPAP |
| | | TIASQPLSLRPEA |
| | | CRPAAGGAVHTRG |
| | | LDFACD |
| IgG1 hinge | SEQ ID NO. 5 | EPKSPDKTHTCPP |
| | | CPAPPVAGPSVFL |
| | | FPPKPKDTLMIAR |
| | | TPEVTCVVVDVSH |
| | | EDPEVKFNWYVDG |
| | | VEVHNAKTKPREE |
| | | QYNSTYRVVSVLT |
| | | VLHQDWLNGKEYK |
| | | CKVSNKALPAPIE |
| | | KTISKAKGQPREP |
| | | QVYTLPPSRDELT |
| | | KNQVSLTCLVKGF |
| | | YPSDIAVEWESNG |
| | | QPENNYKTTPPVL |
| | | DSDGSFFLYSKLT |
| | | VDKSRWQQGNVFS |
| | | CSVMHEALHNHYT |
| | | QKSLSLSPGK |
| CD8α trans- | SEQ ID NO. 6 | IYIWAPLAGTCGV |
| membrane domain | | LLLSLVITLYC |
| 41BB trans- | SEQ ID NO. 7 | IISFFLALTSTAL |
| membrane domain | | LFLLFFLTLRFSV |
| | | V |
| 41BB intra- | SEQ ID NO. 8 | KRGRKKLLYIFKQ |
| cellular domain | | PFMRPVQTTQEED |
| | | GCSCRFPEEEEGG |
| | | CEL |
| CD3ζ intra- | SEQ ID NO. 9 | RVKFSRSADAPAY |
| cellular domain | | QQGQNQLYNELNL |
| | | GRREEYDVLDKRR |
| | | GRDPEMGGKPRRK |
| | | NPQEGLYNELQKD |
| | | KMAEAYSEIGMKG |
| | | ERRRGKGHDGLYQ |
| | | GLSTATKDTYDAL |
| | | HMQALPPR |
| Linker | SEQ ID NO. 10 | GGGGSGGGGSGGG |
| | | GS |

TABLE 2
Sequence of the different
specific CAR components

| | | Raw amino |
|---|---|---|
| ScFv sequences | SEQ ID # | acid sequence |
| 139-heavy chain | SEQ ID NO. 11 | EVQVLESGGGLVQ |
| variable region | | PGGSLRLSCAASG |
| | | FTFSSYAMSWVFQ |
| | | APGKGLEWVSAIS |
| | | GSGGSTNYADSVK |
| | | GRFTISRDNSKNT |
| | | LYLQMNSLRAEDT |
| | | AVYYCAGSSGWSE |
| | | YWGQGTLVTVSS |
| 139-light chain | SEQ ID NO. 12 | DIQMTQSPSSLSA |
| variable region | | SVGDRVTITCRAS |
| | | QGIRNNLAWYQCK |
| | | PGKAPKRLIYAAS |
| | | NLQSGVPSRFTGS |
| | | GSGTEFTLIVSSL |
| | | QPEDFATYYCLQH |
| | | HSYPLTSGGGTKV |
| | | EIK |
| MR1-heavy chain | SEQ ID NO. 13 | QVQLQQSGGGLVK |
| variable region | | PGASLKLSCVTSG |
| | | FTFRKFGMSWVRQ |
| | | TSDKRLEWVASIS |
| | | TGGYNTYYSDNVK |
| | | GRFTISRENAKNT |
| | | LYLQMSSLKSEDT |
| | | ALYYCTRGYSSTS |
| | | YAMDYWGQGTTVT |
| | | V |
| MR1-light chain | SEQ ID NO. 14 | DIELTQSPASLSV |
| variable region | | ATGEKVTIRCMTS |
| | | TDIDDDMNWYQQK |
| | | PGEPPKFLISEGN |
| | | TLRPGVPSRFSSS |
| | | GTGTDFVFTIENT |
| | | LSEDVGDYYCLQS |
| | | ENVPLTEGDGTKL |
| | | EKAL |

TABLE 3
CAR of structure V-1

| | CAR Structure | | | | | | |
|---|---|---|---|---|---|---|---|
| CAR | signal | | | | | | |
| Designation | peptide | | | FcγRIIIα | | | |
| V-1 | (optional) | VH | VL | hinge | CD8α TM | 41BB-IC | CD3ζ CD |
| 139 | SEQ ID | SEQ ID | SEQ ID | SEQ ID | SEQ ID | SEQ ID | SEQ ID |
| (SEQ ID NO. 15) | NO. 1 | NO. 11 | NO. 12 | NO. 3 | NO. 6 | NO. 8 | NO. 9 |
| MR1 | SEQ ID | SEQ ID | SEQ ID | SEQ ID | SEQ ID | SEQ ID | SEQ ID |
| (SEQ ID NO. 17) | NO. 1 | NO. 13 | NO. 14 | NO. 3 | NO. 6 | NO. 8 | NO. 9 |

TABLE 4
CAR of structure V-2

| | CAR Structure | | | | | | |
|---|---|---|---|---|---|---|---|
| CAR | signal | | | | | | |
| Designation | peptide | | | FcγRIIIα | | | |
| V-2 | (optional) | VH | VL | hinge | 41BB-TM | 41BB-IC | CD3ζ CD |
| 139 | SEQ ID | SEQ ID | SEQ ID | SEQ ID | SEQ ID | SEQ ID | SEQ ID |
| (SEQ ID NO. 16) | NO. 1 | NO. 11 | NO. 12 | NO. 3 | NO. 7 | NO. 8 | NO. 9 |
| MR1 | SEQ ID | SEQ ID | SEQ ID | SEQ ID | SEQ ID | SEQ ID | SEQ ID |
| (SEQ ID NO. 18) | NO. 1 | NO. 13 | NO. 14 | NO. 3 | NO. 7 | NO. 8 | NO. 9 |

TABLE 5
CAR of structure V-3

| CAR | CAR Structure | | | | | | |
|---|---|---|---|---|---|---|---|
| Designation | signal | | | CD8α | | | |
| V-3 | peptide | VH | VL | hinge | CD8α TM | 41BB-IC | CD3ζ CD |
| 139 | SEQ ID | SEQ ID | SEQ ID | SEQ ID | SEQ ID | SEQ ID | SEQ ID |
| (SEQ ID NO. 15) | NO. 1 | NO. 11 | NO. 12 | NO. 4 | NO. 6 | NO. 8 | NO. 9 |
| MR1 | SEQ ID | SEQ ID | SEQ ID | SEQ ID | SEQ ID | SEQ ID | SEQ ID |
| (SEQ ID NO. 17) | NO. 1 | NO. 13 | NO. 14 | NO. 4 | NO. 6 | NO. 8 | NO. 9 |

TABLE 6
CAR of structure V-5

| CAR | CAR Structure | | | | | | |
|---|---|---|---|---|---|---|---|
| Designation | signal | | | | | | |
| V-5 | peptide) | VH | VL | IgG1 hinge | CD8α-TM | 41BB-IC | CD3ζ CD |
| 139 | SEQ ID | SEQ ID | SEQ ID | SEQ ID | SEQ ID | SEQ ID | SEQ ID |
| (SEQ ID NO. 16) | NO. 1 | NO. 11 | NO. 12 | NO. 5 | NO. 6 | NO. 8 | NO. 9 |
| MR1 | SEQ ID | SEQ ID | SEQ ID | SEQ ID | SEQ ID | SEQ ID | SEQ ID |
| (SEQ ID NO. 18) | NO. 1 | NO. 13 | NO. 14 | NO. 5 | NO. 6 | NO. 8 | NO. 9 |

The CAR of the invention optionally comprise a linker between VH and VL or between VL and VH, preferably a linker of sequence (G4S)n with n = 1-3, advantageously n = 3

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

TABLE A
General structure of EGFRvIII CAR V-3

| CAR | | | | | | |
|---|---|---|---|---|---|---|
| Designation | CAR Structure | | | | | |
| V-3* | signal | VH and VL | CD8αhinge | CD8αTM | 41BB-IC | CD3ζ CD |
| | peptide | from anti | | | | |
| | | EGFRvIII Ab | | | | |
| V-3* | signal | VH and VL | CD8α | CD8α TM | 41BB-IC | CD3ζ CD |
| | peptide | from anti | hinge | | | |
| | from | EGFRvIII Ab | | | | |
| | CD8α | | | | | |
| V-3 Preferred* | signal | VH and VL | SEQ ID | SEQ ID | SEQ ID | SEQ ID |
| | peptide | from anti | NO. 4 | NO. 6 | NO. 8 | NO. 9 |
| | from | EGFRvIII Ab | | | | |
| | CD8α | | | | | |
| V-3 more | signal | VH and VL | SEQ ID | SEQ ID | SEQ ID | SEQ ID |
| Preferred* | peptide of | from anti | NO. 4 | NO. 6 | NO. 8 | NO. 9 |
| | SEQ ID | EGFRvIII Ab | | | | |
| | NO. 1 | | | | | |

*optionally comprising a linker between VH and VL or between VL and VH, preferably a linker of sequence (G4S)3

TABLE B
General structure of EGFRvIII CAR V-5

| CAR | | | | | | |
|---|---|---|---|---|---|---|
| Designation | CAR Structure | | | | | |
| V-5* | signal | VH and VL | IgG1 hinge | CD8α-TM | 41BB-IC | CD3ζ CD |
| | peptide | from anti | | | | |
| | | EGFRvIII Ab | | | | |
| V-5* | signal | VL and VH | IgG1 hinge | CD8α-TM | 41BB-IC | CD3ζ CD |
| | peptide | from anti | | | | |
| | from | EGFRvIII Ab | | | | |
| | CD8α | | | | | |
| V-5 preferred* | signal | From anti | SEQ ID | SEQ ID | SEQ ID | SEQ ID |
| | peptide | EGFRvIII Ab | NO. 5 | NO. 6 | NO. 8 | NO. 9 |
| | from | | | | | |
| | CD8α | | | | | |

*optionally comprising a linker between VH and VL or between VL and VH, preferably a linker of sequence (G4S)3

EGFRvIII Specific Chimeric Antigen Receptors

The present invention relates to new designs of anti-EGFRvIII chimeric antigen receptor (CAR or EGFRvIII CAR or anti-EGFRvIII CAR) comprising an extracellular ligand-binding domain, a transmembrane domain and a signaling transducing domain.

In general, term “comprises” includes “consists in”, and in a preferred embodiment “comprises” means “consists in”,

In each of the embodiments of the present invention, the term “comprises” can mean consists in.

The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. In a preferred embodiment, said extracellular ligand-binding domain comprises a single chain antibody fragment (scFv) comprising the light (V_{L}) and the heavy (V_{H}) variable fragment of a target antigen specific monoclonal anti EGFRvIII antibody joined by a flexible linker. Said V_{L} and V_{H} are preferably selected from the antibodies referred to as 139 and MR1 as indicated in Table 2. They are preferably linked together by a flexible linker comprising for instance the sequence SEQ ID NO.10. In other words, said CARs preferentially comprise an extracellular ligand-binding domain comprising a polypeptide sequence displaying at least 90%, 95% 97% or 99% identity with an amino acid sequence selected from the group consisting of SEQ ID NO: 11 to SEQ ID NO: 14.

The signal transducing domain or intracellular signaling domain of a CAR according to the present invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector signal function signal and directs the cell to perform a specialized function.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the CAR can comprise the CD3zeta signaling domain which has amino acid sequence with at least 70%, preferably at least 80%, more preferably at least 90%, 95% 97% or 99% sequence identity with amino acid sequence selected from the group consisting of (SEQ ID NO: 9).

In a more preferred embodiment, the intracytoplasmic domain of the EGFRvIII CAR of the invention excludes any sequence from human CD28 or does not comprise a sequence derived from human CD28

In an even more preferred embodiment, the signaling domain of the EGFRvIII CAR comprises a CD3zeta signaling domain which has at least 70%, preferably at least 80%, more preferably at least 90%, even more preferably 95%, 97%, 99% or 100% sequence identity SEQ ID NO: 9 and excludes any sequence from CD28 signaling domain. In particular embodiment the signal transduction domain of the CAR of the present invention comprises a co-stimulatory signal molecule. A co-stimulatory molecule is a cell surface molecule other than an antigen receptor or their ligands that is required for an efficient immune response. “Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1 BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In a preferred embodiment, the signal transduction domain of the CAR of the present invention comprises a part of co-stimulatory signal molecule selected from the group consisting of fragment of 4-1BB (GenBank: AAA53133.) and CD28 (NP_006130.1). In particular the signal transduction domain of the CAR of the present invention comprises amino acid sequence which comprises at least 70%, preferably at least 80%, more preferably at least 90%, 95% 97% or 99% sequence identity with amino acid sequence selected from the group consisting of SEQ ID NO: 8.

In a preferred embodiment the signal transduction domain of the EGFRvIII CAR of the present invention comprises a co-stimulatory signal molecule from 4 1BB and excludes any co-stimulatory signal molecule from human CD28.

A CAR according to the present invention is expressed on the surface membrane of the cell. Thus, such CAR further comprises a transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T-cell receptor such as α, β, γ or δ, polypeptide constituting CD3 complex, IL2 receptor p55 (α chain), p75 (β chain) or γ chain, subunit chain of Fc receptors, in particular Fcγ receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1)

In a preferred embodiment the TM domain of the EGFRvIII CAR of the present invention is derived from the CD8 alpha chain (e.g. NP_001139345.1)

The transmembrane domain can further comprise a hinge region between said extracellular ligand-binding domain and said transmembrane domain. The term “hinge region” used herein generally means any oligo- or polypeptide that functions to link the transmembrane domain to the extracellular ligand-binding domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively the hinge region may be a synthetic sequence that corresponds to a naturally occurring hinge sequence, or may be an entirely synthetic hinge sequence. In a preferred embodiment said hinge domain comprises a part of human CD8 alpha chain, FcγRIIIα receptor or IgG1 respectively referred to in this specification as SEQ ID NO. 3, SEQ ID NO. 4 and SEQ ID NO.5, or hinge polypeptides which display preferably at least 80%, more preferably at least 90%, 95% 97% or 99% sequence identity with these polypeptides.

In preferred embodiment the EGFRvIII CAR of the present invention comprises a hinge from CD8α of SEQ ID NO. 4, a TM domain from CD8α and a peptide signal from CD8α.

In a more preferred embodiment the EGFRvIII CAR of the present invention comprises a CD8α hinge which display preferably at least 80%, more preferably at least 90%, 95% 97% or 99% sequence identity with of SEQ ID NO. 4, a TM domain from CD8α and a peptide signal from CD8α.

A car according to the invention generally further comprises a transmembrane domain (TM) more particularly selected from CD8α and 4-1BB, showing identity with the polypeptides of SEQ ID NO. 6 or 7.

Preferably, an EGFRvIII CAR according to the invention comprises a TM showing at least 70%, preferably at least 80%, more preferably at least 90%, 95% 97%, 99% or 100% sequence identity with the polypeptides of SEQ ID NO. 6.

Also disclosed herein are EGFRvIII CAR constructs of the invention wherein the hinge region is a hinge region from CD8α. For example, CAR constructs of the invention may combine the hinge region derived from CD8 α with a signal peptide derived from CD8 α and/or a transmembrane region also derived from CD8 α.

In a preferred embodiment, EGFRvIII CAR constructs of the invention may combine the hinge region from CD8 α with a signal peptide from CD8 α and a transmembrane region also derived from CD8 α.

In an even more preferred embodiment, CAR constructs of the invention may combine the hinge region from CD8 α with a signal peptide derived from CD8 α and a transmembrane region also derived from CD8 α and excludes any sequence from CD28 signaling domain.

Downregulation or mutation of target antigens is commonly observed in cancer cells, creating antigen-loss escape variants. Thus, to offset tumor escape and render immune cell more specific to target, the EGFRvIII specific CAR according to the invention can comprise another extracellular ligand-binding domains, to simultaneously bind different elements in target thereby augmenting immune cell activation and function. In one embodiment, the extracellular ligand-binding domains can be placed in tandem on the same transmembrane polypeptide, and optionally can be separated by a linker.

In another embodiment, said different extracellular ligand-binding domains can be placed on different transmembrane polypeptides composing the CAR.

In another embodiment, the present invention relates to a population of CARs comprising each one different extracellular ligand binding domains. In a particular, the present invention relates to a method of engineering immune cells comprising providing an immune cell and expressing at the surface of said cell a population of CAR each one comprising different extracellular ligand binding domains. In another particular embodiment, the present invention relates to a method of engineering an immune cell comprising providing an immune cell and introducing into said cell polynucleotides encoding polypeptides composing a population of CAR each one comprising different extracellular ligand binding domains. By population of CARs, it is meant at least two, three, four, five, six or more CARs each one comprising different extracellular ligand binding domains. The different extracellular ligand binding domains according to the present invention can preferably simultaneously bind different elements in target thereby augmenting immune cell activation and function. The present invention also relates to an isolated immune cell which comprises a population of CARs each one comprising different extracellular ligand binding domains.

The present invention provides an EGFRVIII specific chimeric antigen receptor (EGFRVIII CAR) comprising:
a binding domain specific for EGFRVIII, preferably a binding domain specific for human EGFRVIII, more preferably said binding domain specific for human EGFRVIII is a single-chain variable fragment (scFv).
a hinge,
a transmembrane domain,
a co-stimulatory signal molecule from human 4-1 BB, and
   an intracellular signaling domain comprising a human CD3zeta signaling domain.

In one embodiment the EGFRVIII CAR of the invention has no sequence from human CD28.

In a preferred embodiment, the EGFRVIII CAR of the invention does not include a CD28-derived sequence, in particular has no sequence having at least 1, at least 2, at least 3, at least 4, at least 5, at least 6, at least 7 at least 8, at least 9 at least 10 amino acids identity with human CD28.

In a more preferred embodiment, the intracytoplasmic and/or transmembrane domain of the EGFRVIII CAR of the invention does not include human CD28-derived sequence, in particular has no sequence having at least 1, at least 2 at least 3, at least 4 at least 5, at least 6, at least 7 at least 8, at least 9 at least 10 amino acids identity with human CD28.

The EGFRVIII CAR of the invention comprises:
a binding domain specific for EGFRVIII, preferably a binding domain specific for human EGFRVIII, more preferably said binding domain specific for human EGFRVIII is a single-chain variable fragment (scFv),
a hinge,
a transmembrane domain,
a co-stimulatory signal molecule from human 4-1 BB,
an intracellular signaling domain consisting in a human CD3zeta signaling domain and no human CD28 signaling domain.

In a preferred embodiment, the EGFRVIII CAR of the invention does not contain any sequence from CD28 and comprises a signal peptide (or leader sequence), a TM domain and a hinge from CD8 α.

In one embodiment, the EGFRVIII CAR of the invention comprises a leader sequence from human CD8 α (SEQ ID NO.1.) or a leader sequence having at least 90%, at least 91%, at least 92%, at least 93% at least 94%, at least 95%, at least 96%, at least 97%, at least 98%, at least 99% or having 100% identity with SEQ ID NO.1, preferably having 100% identity with SEQ ID NO. 1.

In another embodiment, The EGFRVIII CAR of the invention comprises a leader sequence of SEQ ID NO.2 or a leader sequence having at least 90%, at least 91%, at least 92%, at least 93% at least 94%, at least 95%, at least 96%, at least 97%, at least 98%, at least 99% or having 100% identity with SEQ ID NO.2, preferably having 100% identity with SEQ ID NO. 2.

In one embodiment the present invention provides an EGFRVIII specific chimeric antigen receptor (EGFRVIII CAR) comprising:
a binding domain specific for EGFRVIII, preferably a domain specific for human EGFRVIII, more preferably said domain specific for human EGFRVIII is a single-chain variable fragment (scFv),
a hinge from human CD8 alpha chain (from CD8 α)
a transmembrane domain from human CD8alpha(α)
a co-stimulatory signal molecule from human 4-1 BB
an intracellular signaling domain comprising a human CD3zeta signaling domain.

The present invention provides an EGFRVIII specific chimeric antigen receptor (EGFRVIII CAR) comprising:
a binding domain specific for EGFRVIII, preferably a domain specific for human EGFRVIII, more preferably said domain specific for human EGFRVIII is a single-chain variable fragment (scFv).
a hinge from human IgG1
a transmembrane domain from human CD8alpha(α)
a co-stimulatory signal molecule from human 4-1 BB
an intracellular signaling domain comprising a human CD3zeta signaling domain.

The present invention encompasses an EGFRVIII CAR of the invention with a signal peptide of SEQ ID NO 1 or of SEQ ID NO 2.

In the present invention, an scfv is a fusion protein of the variable regions of the heavy (V_{H domain}) and light chains (V_{L domain}) Or a V_{L domain} with a V_{H domain}) of an immunoglobulin specific for EGFRVIII, preferably connected with a short linker peptide of 4 to 25 amino acids, more preferably of SEQ ID NO. 10.

The scfv of the invention is derived from an antibody specific for EGFRVIII, it comprises a VH domain separated to a VL domain by a linker, said VH and/or VL domains, together contributing to the binding to EGFRVIII.

In one embodiment, said scfv of the invention further comprises a leader sequence (or signal peptide), preferably said leader sequence is linked to the VH domain.

An embodiment wherein said leader sequence is linked to the VL domain is part of the present invention.

Preferably said leader sequence is having an amino acid sequence of SEQ ID NO. 1 or 2, more preferably of SEQ ID NO. 1.

In one embodiment, a VH domain is linked to a hinge, in another embodiment a VL domain is linked to said hinge.

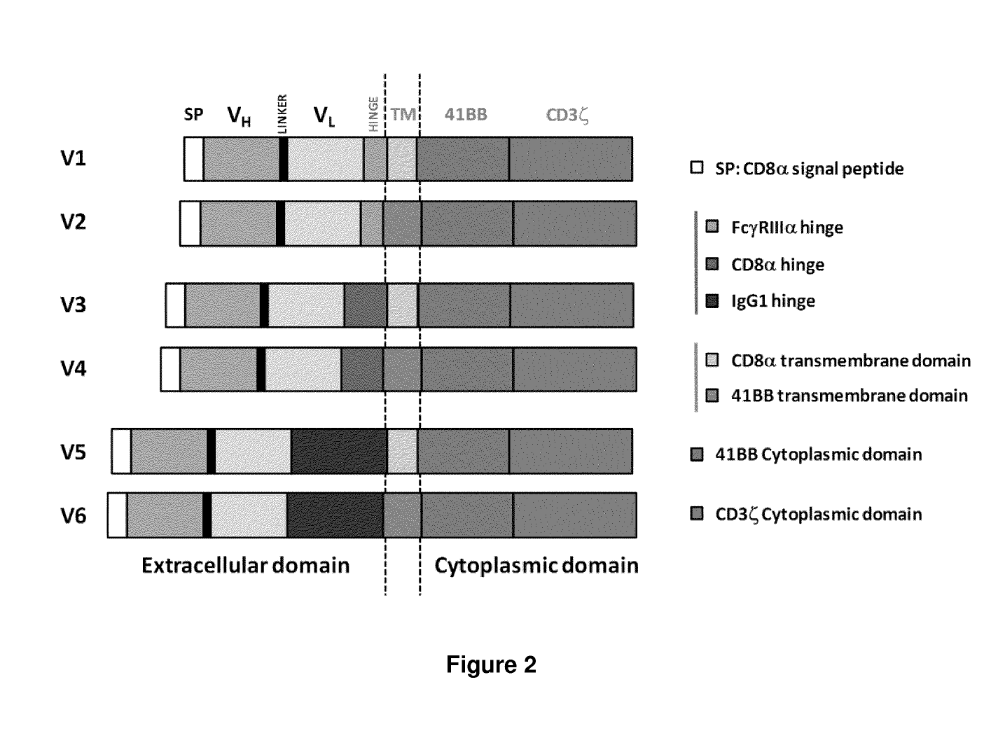

The present invention provides anti-EGFRVII scfv linked to a hinge having different length preferably a hinge from CD8α, IgG1 or FCRIII (See FIG. 2), more preferably a hinge from CD8α, even more preferably a hinge with a SEQ ID NO.4.

Preferably, the present invention provides an EGFRVIII CAR comprising:
a signal peptide, preferably a signal peptide from CD8alpha, more preferably a signal peptide from CD8alpha of SEQ ID NO. 1 or of SEQ ID NO. 2.
a (scFv) comprising a VH domain separated to a VL domain by a linker, said VH and VL contributing to the binding to EGFRVIII,
a hinge from human CD8 alpha chain or a Hinge from human IgG1
a transmembrane domain (TM) from CD8alpha(α)
a co-stimulatory signal molecule from human 4-1 BB
an intracellular signaling domain comprising the CD3zeta signaling domain.

More preferably, the present invention provides an EGFRVIII CAR comprising:
a signal peptide from human CD8alpha,
a (scFv) comprising a VH domain separated to a VL domain by a linker, said VH and VL contributing to the binding to EGFRVIII,
a hinge from human CD8 alpha chain or a Hinge from human IgG1
a transmembrane domain (TM) from CD8alpha(α)
a co-stimulatory signal molecule from human 4-1 BB
an intracellular signaling domain comprising the CD3zeta signaling domain.

Even more preferably, the present invention provides an EGFRVIII CAR comprising:
a signal peptide from human CD8alpha,
a (scFv) comprising a VH domain separated to a VL domain by a linker, said VH and VL contributing to the binding to EGFRVIII and said VH and VL domains being humanized
a hinge from human CD8 alpha chain or a Hinge from human IgG1
a transmembrane domain (TM) from human CD8alpha(α)
a co-stimulatory signal molecule from human 4-1 BB
an intracellular signaling domain comprising the human CD3zeta signaling domain.

And even more preferably, the EGFRVIII CAR of the invention comprises:
a leader sequence (for example a CD8 α leader sequence or a CD8α signal peptide)
an anti-EGFRVIII scfv comprising a VH, a linker, and a VL,
   a CD8 α hinge
   a CD8 α TM
   a co-stimulatory signal molecule from 4-1 BB
   an intracellular CD3zeta signaling domain.

And even more preferably, the EGFRVIII CAR of the invention comprises:
a leader sequence (for example a CD8 α leader sequence or a CD8α signal peptide)
an anti-EGFRVIII scfv comprising a VL, a linker, and a VH,
   a CD8 α hinge
   a CD8 α TM
   a co-stimulatory signal molecule from 4-1 BB
   an intracellular CD3zeta signaling domain.

And even more preferably, the EGFRVIII CAR of the invention consists in:
a leader sequence (for example a CD8 α leader sequence or a CD8α signal peptide)
an anti-EGFRVIII scfv comprising a VH, a linker, and a VL,
   a CD8 α hinge
   a CD8 α TM
   a co-stimulatory signal molecule from 4-1 BB
   an intracellular CD3zeta signaling domain

In one embodiment, said linker is a linker of formula (G4S)n wherein n is 1 to 3; preferably n=3 and said sequence is (G4S)3, more preferably of SEQ ID NO. 10.

One EGFRVIII CAR of the invention consists in:
a human CD8α leader sequence (CD8 α leader or CD8α signal peptide)
an anti-EGFRVIII scfv comprising a VH, a linker, and a VL, advantageously the scfv is humanized,
   a human CD8 α hinge
   a human CD8 α TM
   a co-stimulatory signal molecule from 4-1 BB
   an intracellular CD3zeta signaling domain.

In one embodiment the present invention provides:

An EGFRVIII CAR comprising:
a human CD8α leader sequence (CD8 α leader or CD8α signal peptide) of SEQ ID NO. 1
an anti-EGFRVIII scfv comprising a VH of SEQ ID NO. 11, a linker of SEQ ID No 10, and a VL of SEQ ID NO 12 or a VH of SEQ ID NO. 13, a linker of SEQ ID No 10 and a VL of SEQ ID NO 14, advantageously the scfv is humanized,
   a human CD8 α hinge of SEQ ID NO.4,
   a human CD8 α TM of SEQ ID NO.6
   a co-stimulatory signal molecule from 4-1BB of SEQ ID NO.8
   an intracellular CD3zeta signaling domain of SEQ ID NO. 9.

In one embodiment, the present invention provides:

An EGFRVIII CAR comprising
a human CD8α leader sequence (CD8 α leader or CD8α signal peptide) of SEQ ID NO. 1
   an anti-EGFRVIII scfv comprising a VL of SEQ ID NO 12, a linker of SEQ ID No 10, and a VH of SEQ ID NO. 11, a VL of SEQ ID NO 14, a linker of SEQ ID No 10 and a VH of SEQ ID NO. 13, advantageously the scfv is humanized.
a human CD8 α hinge of SEQ ID NO.4,
a human CD8 α TM of SEQ ID NO.6
a co-stimulatory signal molecule from 4-1BB of SEQ ID NO.8
an intracellular CD3zeta signaling domain of SEQ ID NO. 9.

In one embodiment, the present invention provides an EGFRVIII specific chimeric antigen receptor (EGFRVIII CAR) comprising:
a signal peptide having an amino acid sequence with at least 80%, more preferably at least 90%, 95% 97%, 99% or 100% sequence identity with the polypeptide of SEQ ID NO. 1 or 2; preferably the signal peptide has an amino acid sequence with at least 80%, more preferably at least 90%, 95% 97%, 99% or 100% sequence identity with the polypeptide of SEQ ID NO 1.
a VH domain separated to a VL domain by a linker, said VH and VL contributing to the binding to EGFRVIII; said linker having at least 90%, 95% 97%, 99% or 100% sequence identity with the polypeptide of SEQ ID NO 10.
   Said VH domain having at least 90%, 95% 97%, 99% or 100% sequence identity with the polypeptide of SEQ ID NO 11 or SEQ ID NO 13
   Said VL domain having at least 90%, 95% 97%, 99% or 100% sequence identity with the polypeptide of SEQ ID NO 12 or SEQ ID NO 14.
a hinge derived from human CD8 alpha chain having an amino acid sequence with at least 80%, more preferably at least 90%, 95% 97%, 99% or 100% sequence identity with the polypeptide of SEQ ID NO. 4;
a transmembrane domain derived from CD8alpha(α) having an amino acid sequence with at least 90%, 91%, 92%, 93%, 94%, 95%, 96%, 97%, 98%, 99% or 100% identity with the polypeptide of SEQ ID NO. 6;
a co-stimulatory signal molecule derived from human 4-1 BB (or 4-1 BB intracellular domain) having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9.

In a preferred embodiment, the EGFRVIII specific chimeric antigen receptor (EGFRVIII CAR) of the present invention does not comprise any sequence from CD28 or from human CD28, in particular from human CD28 intra signaling domain. In a more preferred embodiment, the EGFRVIII specific chimeric antigen receptor (EGFRVIII CAR) of the present invention does not comprise any sequence from human CD28, in particular from human CD28 intra signaling domain and further contains a signal peptide from CD8α, preferably fused to the VH domain of a scfv specific for EGFRVIII.

In one embodiment, the present invention provides an EGFRVIII CAR of SEQ ID NO. 24.

In one embodiment the present invention provides an EGFRVIII CAR of SEQ ID NO. 25.

In one embodiment the present invention provides an EGFRVIII CAR of SEQ ID NO. 26.

In one embodiment the present invention provides an EGFRVIII CAR of SEQ ID NO. 27.

In a preferred embodiment the present invention provides an EGFRVIII CAR of SEQ ID NO. 24 or of SEQ ID NO. 25, more preferably of SEQ ID NO. 24.

In one embodiment, the present invention provides an EGFRVIII CAR having an amino acid sequence with at least 90%, 91%, 92%, 93%, 94%, 95%, 96%, 97%, 98%, 99% or 100% identity with the polypeptide of SEQ ID No 24.

In one embodiment the present invention provides an EGFRVIII CAR having an amino acid sequence with at least 90%, 91%, 92%, 93%, 94%, 95%, 96%, 97%, 98%, 99% or 100% identity with the polypeptide of SEQ ID No 25.

In one embodiment the present invention provides an EGFRVIII CAR having an amino acid sequence with at least 90%, 91%, 92%, 93%, 94%, 95%, 96%, 97%, 98%, 99% or 100% identity with the polypeptide of SEQ ID No 26.

In one embodiment the present invention provides an EGFRVIII CAR having an amino acid sequence with at least 90%, 91%, 92%, 93%, 94%, 95%, 96%, 97%, 98%, 99% or 100% identity with the polypeptide of SEQ ID No 27.

In one aspect, the anti-EGFRvIII binding domain of the EGFRVIII CAR of the invention is a humanized anti-EGFRvIII binding domain.

Any of the anti-EGFRvIII CAR of the invention may be a humanized anti-EGFRvIII binding domain with amino acid modifications that do not significantly affect or alter the binding characteristics of the CAR and/or that do not significantly affect the activity of the CAR T cell containing the modified amino acid sequence and reduce or abolish a human anti-mouse antibody (HAMA) response.

“Humanization” is intended to refer to amino acid modifications that do not significantly affect or alter the binding characteristics of the CAR and/or that do not significantly affect the activity of the CAR T cell containing the modified amino acid sequence and reduce or abolish a human anti-mouse antibody (HAMA) response.

“Humanization” is intended to refer to amino acid modifications that may significantly improve the binding characteristics (affinity avidity) of the CAR and/or that do not significantly affect the activity of the CAR T Cell containing the modified amino acid sequence and reduce or abolish a human anti-mouse antibody (HAMA) response.

Such conservative modifications include amino acid substitutions, additions and deletions in said antibody fragment in said CAR and/or any of the other parts of said CAR molecule. Modifications can be introduced into an antibody, into an antibody fragment or in any of the other parts of the CAR molecule of the invention by standard techniques known in the art, such as site-directed mutagenesis, PCR-mediated mutagenesis or by employing optimized germline sequences.

The term “conservative sequence modifications” or “amino acid change” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

In a preferred embodiment, the present invention provides an EGFRVIII CAR having conservative sequence modifications (or an amino acid sequence change) as compared to the amino acid sequence of the polypeptide of SEQ ID No 24.

In a preferred embodiment, the present invention provides an EGFRVIII CAR having an amino acid sequence with 2 amino acid changes as compared to the amino acid sequence of the polypeptide of SEQ ID No 24.

In a preferred embodiment, the present invention provides an EGFRVIII CAR having an amino acid sequence with 3 amino acid changes as compared to the amino acid sequence of the polypeptide of SEQ ID No 24.

In a preferred embodiment, the present invention provides an EGFRVIII CAR having an amino acid sequence with 4 amino acid changes as compared to the amino acid sequence of the polypeptide of SEQ ID No 24.

In a preferred embodiment, the present invention provides an EGFRVIII CAR having an amino acid sequence with 5 amino acid changes as compared to the amino acid sequence of the polypeptide of SEQ ID No 24.

In a more preferred embodiment, the present invention provides an EGFRVIII CAR having an amino acid sequence with 5 amino acid changes as compared to the amino acid sequence of the polypeptide of SEQ ID No 24 and all CDR in SEQ ID No 24 are conserved.

In a more preferred embodiment, the present invention provides an EGFRVIII CAR having an amino acid sequence with from 1 to 15 amino acid changes as compared to the amino acid sequence of the polypeptide of SEQ ID No 24 and all CDR in SEQ ID No 24 are conserved.

In a preferred embodiment, the sequence of EGFRVIII CAR of the invention is modified by changing at least 1 amino acid, from 2 to 15 amino acids as compared to SEQ ID NO 24, to reduce the HAMA (human anti-mouse response), without modifying the binding capacity of said CAR to its target (EGFRVIII). In one embodiment, said binding may be improved.

In a preferred embodiment, the present invention provides an EGFRVIII CAR having an amino acid sequence with at least 1 amino acid change as compared to the amino acid sequence of the polypeptide of SEQ ID No 24 said at least 1 amino acid change having no impact or improving the binding and/or activity of said EGFRVIII CAR in primary T cells.

The invention also provides related nucleic acids, recombinant expression vectors, host cells, populations of cells, and pharmaceutical compositions relating to the EGFRVIII CARs of the invention.

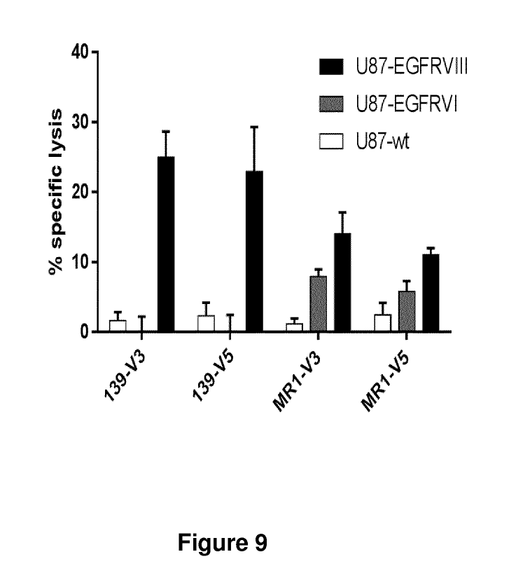

Also disclosed herein are EGFRVIII CAR constructs of the invention wherein the hinge, when combined to the TM domain and signal peptide of CD8α, is conferring a better affinity and selectivity of said EGFRVIII CAR to EGFRVIII expressing cells (as seen in FIG. 9) as compared to previous CAR constructs that do not combined these structural elements and technical features. Preferably, EGFRVIII CAR constructs of the invention with a better affinity and selectivity for EGFRVIII expressing cells combine technical features of the V3 architecture, more preferably EGFRVIII CAR constructs of the invention with a better affinity and selectivity for EGFRVIII expressing cells have a sequence having at least 80% identity with of SEQ ID NO. 24 and are humanized.

Also disclosed herein are EGFRVIII CAR constructs of the invention wherein the structure is conferring a better affinity and selectivity of said EGFRVIII CAR to EGFRVIII expressing cells (as seen in FIG. 9) as compared to previous CAR constructs that do not combined these structural elements and technical features.

Preferably, EGFRVIII CAR constructs of the invention with a better affinity and selectivity for EGFRVIII expressing cells combine technical features of the V3 architecture, more preferably EGFRVIII CAR constructs of the invention with a better affinity and selectivity for EGFRVIII expressing cells have a sequence having at least 80% identity with of SEQ ID NO. 24 and are humanized.

### Polynucleotides, Vectors:

The present invention also relates to polynucleotides, vectors encoding the above described CAR according to the invention.

The polynucleotide may consist in an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell).

In a particular embodiment, the different nucleic acid sequences can be included in one polynucleotide or vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

A vector allowing an EGFRVIII CAR of the invention to be expressed in a cell is another object of the present invention. In a preferred embodiment, said vector allows a transient expression of the EGFRVIII CAR of the invention. In a more preferred embodiment said vector allows a constitutive and stable expression of the EGFRVIII CAR of the invention by insertion of the sequence into the genome of a cell. The expression of the EGFRVIII CAR of the invention and/or the survival of the cell expressing the EGFRVIII CAR of the invention may be controlled.

In one embodiment, the present invention provides a vector comprising a sequence coding an EGFRVIII CAR of the invention.

In a preferred embodiment, the present invention provides a vector comprising a sequence coding an EGFRVIII CAR of the invention selected from SEQ ID NO. 24, SEQ ID NO. 25, SEQ ID NO. 26 and SEQ ID NO. 27.

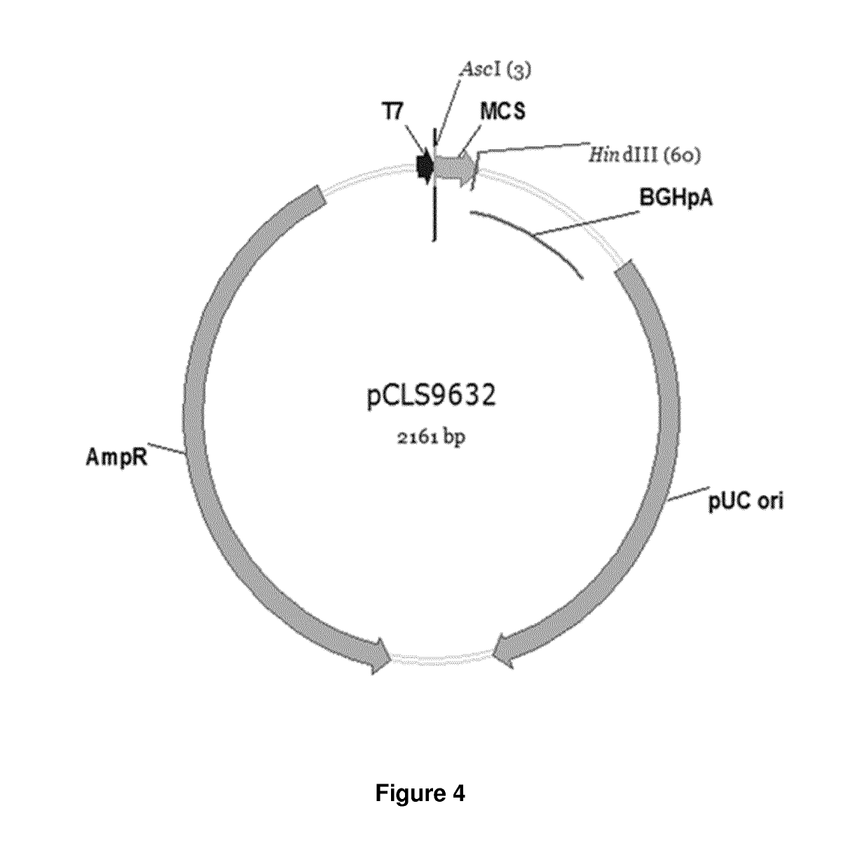

In a more preferred embodiment, the present invention provides a pCLS 9632 vector (as in FIG. 4) comprising a sequence coding a CAR of the invention.

In a more preferred embodiment, the present invention provides a pCLS 9632 vector (as in FIG. 4) comprising a sequence coding a CAR selected from SEQ ID NO. 24, SEQ ID NO. 25, SEQ ID NO. 26 and SEQ ID NO. 27.

In one embodiment, the present invention provides a pCLS 9632 vector (as in FIG. 4) comprising a sequence coding a CAR of SEQ ID NO. 25.

In a more preferred embodiment, the present invention provides a pCLS 9632 vector (as in FIG. 4) comprising a sequence coding a CAR of SEQ ID NO. 27.

In a more preferred embodiment, the present invention provides a pCLS 9632 vector (as in FIG. 4) comprising a sequence coding a CAR of SEQ ID NO. 26.

In an even more preferred embodiment, the present invention provides a pCLS 9632 vector (as in FIG. 4) comprising a sequence coding a CAR of SEQ ID NO. 24.

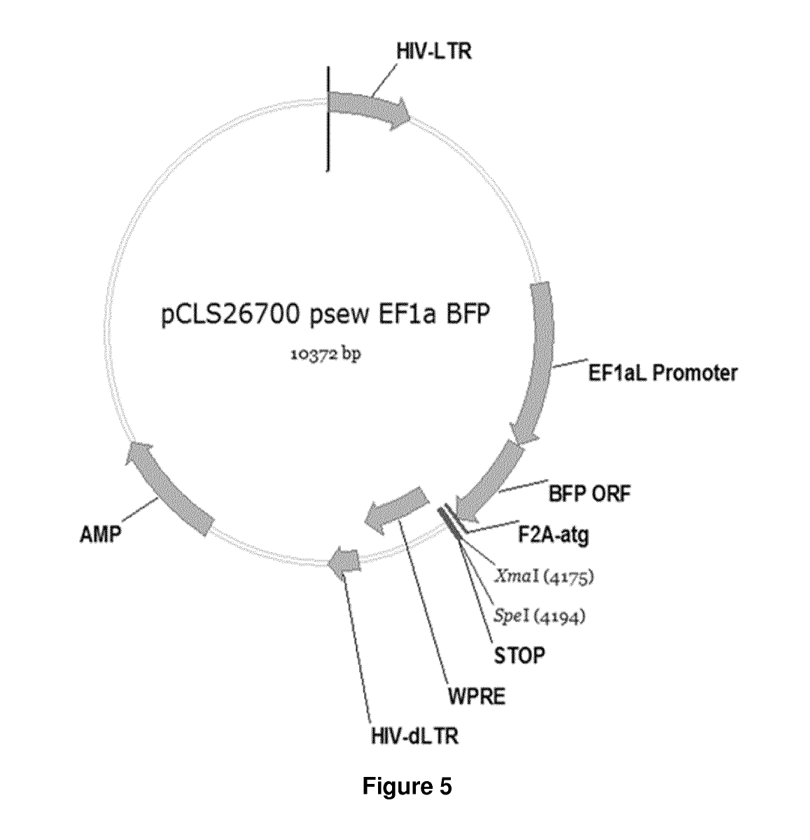

In another embodiment the present invention provides a pCLS 26700 vector (as in FIG. 5) comprising a CAR of the invention.

In another embodiment the present invention provides a pCLS 26700 vector (as illustrated in FIG. 5) comprising a CAR sequence coding a CAR selected from SEQ ID NO. 24, SEQ ID NO. 25, SEQ ID NO. 26 and SEQ ID NO. 27, said CAR being optionally humanized.

In another more preferred embodiment the present invention provides a pCLS 26700 vector (as in FIG. 5) comprising a sequence coding a CAR of SEQ ID NO. 24, In another more preferred embodiment the present invention provides a pCLS 26700 vector (as in FIG. 5) comprising a sequence coding a CAR of SEQ ID NO. 25,

In another more preferred embodiment the present invention provides a pCLS 26700 vector (as in FIG. 5) comprising a sequence coding a CAR of SEQ ID NO. 26,

In another more preferred embodiment the present invention provides a pCLS 26700 vector (as in FIG. 5) comprising a sequence coding a CAR of SEQ ID NO. 27,

To direct transmembrane polypeptide into the secretory pathway of a host cell, a secretory signal sequence (also known as a leader sequence, prepro sequence or pre sequence) is provided in polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). In a preferred embodiment the signal peptide comprises the amino acid sequence SEQ ID NO: 1 and 2.

In a more preferred embodiment, the signal peptide of the CAR of the invention comprises the amino acid sequence of SEQ ID NO: 1.

Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

Methods of Engineering Immune Cells Endowed with CARs:

The present invention encompasses the method of preparing immune cells for immunotherapy comprising introducing ex-vivo into said immune cells the polynucleotides or vectors encoding one of the EGFRvIII CAR as previously described.

The present invention also encompasses primary immune cells comprising an immune cell endowed a polynucleotides or lentiviral vectors encoding one of the EGFRvIII CAR of the invention, preferably for immunotherapy, more preferably more therapy of cancer.

The primary immune cells of the invention comprise EGFRVIII CAR constructs of the invention with a better affinity and selectivity for EGFRVIII expressing cancer cells.

In a preferred embodiment, said polynucleotides are included in lentiviral vectors (preferably as in FIG. 5) in view of being stably expressed in the immune cells.

According to further embodiments, said method further comprises the step of genetically modifying said cell to make them more suitable for allogeneic transplantation.

According to a first aspect, the immune cell can be made allogeneic, for instance, by inactivating at least one gene expressing one or more component of T-cell receptor (TCR) as described in WO 2013/176915, which can be combined with the inactivation of a gene encoding or regulating HLA or β2m protein expression. Accordingly the risk of graft versus host syndrome and graft rejection is significantly reduced.

According to another aspect, the immune cells can be further genetically engineered to improve their resistance to immunosuppressive drugs or chemotherapy treatments, which are used as standard care for treating EGFRvIII positive malignant cells. For instance, CD52 and glucocorticoid receptors (GR), which are drug targets of Campath (alemtuzumab) and glucocorticoids treatments, can be inactivated to make the cells resistant to these treatments and give them a competitive advantage over patient's own T-cells not endowed with specific EGFRvIII CARs. Expression of CD3 gene can also be suppressed or reduced to confer resistance to Teplizumab, which is another immune suppressive drug. Expression of HPRT can also be suppressed or reduced according to the invention to confer resistance to 6-thioguanine, a cytostatic agent commonly used in chemotherapy especially for the treatment of acute lymphoblasic leukemia.

According to further aspect of the invention, the immune cells can be further manipulated to make them more active or limit exhaustion, by inactivating genes encoding proteins that act as “immune checkpoints” that act as regulators of T-cells activation, such as PDCD1 or CTLA-4. Examples of genes, which expression could be reduced or suppressed are indicated in Table 9.

TABLE 9
List of genes encoding immune checkpoint proteins.

| | | Genes that can be inactivated |
|---|---|---|
| Pathway | | In the pathway |
| Co-inhibitory | CTLA4 (CD152) | CTLA4, PPP2CA, PPP2CB, PTPN6, |
| receptors | | PTPN22 |
| | PDCD1 (PD-1, CD279) | PDCD1 |
| | CD223 (lag3) | LAG3 |
| | HAVCR2 (tim3) | HAVCR2 |
| | BTLA(cd272) | BTLA |
| | CD160(by55) | CD160 |
| | IgSF family | TIGIT |
| | | CD96 |
| | | CRTAM |
| | LAIR1(cd305) | LAIR1 |
| | SIGLECs | SIGLEC7 |
| | | SIGLEC9 |
| | CD244(2b4) | CD244 |
| Death receptors | TRAIL | TNFRSF10B, TNFRSF10A, CASP8, |
| | | CASP10, CASP3, CASP6, CASP7 |
| | FAS | FADD, FAS |
| Cytokine signalling | TGF-beta signaling | TGFBRII, TGFBRI, SMAD2, SMAD3, |
| | | SMAD4, SMAD10, SKI, SKIL, TGIF1 |
| | IL10 signalling | IL10RA, IL10RB, HMOX2 |
| | IL6 signalling | IL6R, IL6ST |
| Prevention of TCR | | CSK, PAG1 |
| signalling | | SIT1 |
| Induced Treg | induced Treg | FOXP3 |
| Transcription | transcription factors | PRDM1 (=blimp1, heterozygotes mice |
| factors controlling | controlling exhaustion | control chronic viral infection better |
| exhaustion | | than wt or conditional KO) |
| | | BATF |
| Hypoxia mediated | iNOS induced guanylated | GUCY1A2, GUCY1A3, GUCY1B2, |
| tolerance | cyclase | GUCY1B3 |

In a preferred embodiment said method of further engineering the immune cells involves introducing into said T cells polynucleotides, in particular mRNAs, encoding specific rare-cutting endonuclease to selectively inactivate the genes, as those mentioned above, by DNA cleavage. In a more preferred embodiment said rare-cutting endonucleases are TALE-nucleases or Cas9 endonuclease. TAL-nucleases have so far proven higher specificity and cleavage efficiency over the other types of rare-cutting endonucleases, making them the endonucleases of choice for producing of the engineered immune cells on a large scale with a constant turn-over.

Delivery Methods

The different methods described above involve introducing CAR into a cell. As non-limiting example, said CAR can be introduced as transgenes encoded by one plasmid vector. Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

Engineered Immune Cells

A primary immune cell endowed with an EGFRVIII CAR of the invention (or engineered immune cells) is another object of the present invention. Preferably said cell is a primary T cell, more preferably a primary T cell having a CTL activity towards EGFRVIII expressing cells resulting in the destruction of EGFRVIII expressing cells.

Preferably, said primary T cell is endowed with an EGFRVIII CAR of SEQ ID NO. 24.
Preferably, said primary T cell is endowed with an EGFRVIII CAR of SEQ ID NO. 25.
Preferably, said primary T cell is endowed with an EGFRVIII CAR of SEQ ID NO. 26.
Preferably, said primary T cell is endowed with an EGFRVIII CAR of SEQ ID NO. 27, more preferably said primary T cell is endowed with an EGFRVIII CAR of SEQ ID NO. 24, optionally humanized.

The present invention provides a primary T cell expressing an EGFRVIII CAR of the invention and exhibiting a CTL and/or degranulating activity towards an EGFRVIII-expressing cell, preferably towards an EGFRVIII-expressing cancer cell, preferably, said primary T cell exhibiting a CTL and/or degranulating activity towards an EGFRVIII-expressing cell, preferably towards an EGFRVIII-expressing cancer cell, is endowed with an EGFRVIII CAR of SEQ ID NO. 27, more preferably said primary T cell is endowed with an EGFRVIII CAR of SEQ ID NO. 24, optionally humanized.

The present invention also provides a primary T cell expressing an EGFRVIII CAR of the invention for lysing an EGFRVIII-expressing cell, in particular an EGFRVIII-expressing tumor cell.

Also disclosed herein are primary T cells expressing an EGFRVIII CAR of the invention and exhibiting a CTL and/or degranulating activity towards an EGFRVIII-expressing cell, preferably towards an EGFRVIII-expressing cancer cell,

Also disclosed herein are primary T cells expressing an EGFRVIII CAR of the invention and exhibiting a CTL and/or degranulating activity towards an EGFRVIII-expressing cell, preferably towards an EGFRVIII-expressing cancer cell,

The following additional and specific subject matter is also provided:

Primary immune T cells of the invention expressing an EGFRVIII CAR comprising:
a binding domain specific for EGFRVIII, preferably a binding domain specific for human EGFRVIII, more preferably said binding domain specific for human EGFRVIII is a single-chain variable fragment (scFv).
a hinge,
a transmembrane domain,
a co-stimulatory signal molecule from human 4-1 BB, and
   an intracellular signaling domain comprising a human CD3zeta signaling domain.

In one embodiment the EGFRVIII CAR expressed in primary immune T cells of the invention has no sequence from human CD28.

In a preferred embodiment, the primary immune T cells of the invention express an EGFRVIII CAR comprising: no CD28-derived sequence, in particular has no sequence having at least 1, at least 2, at least 3, at least 4, at least 5, at least 6, at least 7 at least 8, at least 9 at least 10 amino acids identity with human CD28.

In a more preferred embodiment, the intracytoplasmic and/or transmembrane domain of the EGFRVIII CAR of the invention in the primary immune T cells of the invention, does not include human CD28-derived sequence, in particular has no sequence having at least 1, at least 2 at least 3, at least 4 at least 5, at least 6, at least 7 at least 8, at least 9 at least 10 amino acids identity with human CD28.

Primary immune T cells of the invention express an EGFRVIII CAR comprising:
a binding domain specific for EGFRVIII, preferably a binding domain specific for human EGFRVIII, more preferably said binding domain specific for human EGFRVIII is a single-chain variable fragment (scFv),
a hinge,
a transmembrane domain,
a co-stimulatory signal molecule from human 4-1 BB,
an intracellular signaling domain consisting in a human CD3zeta signaling domain and no human CD28 signaling domain.

In a preferred embodiment, primary immune T cells of the invention express an EGFRVIII CAR comprising: no sequence from CD28 and a signal peptide (leader sequence), a TM domain and a hinge from CD8 α.

In one embodiment, primary immune T cells of the invention express an EGFRVIII CAR comprising a leader sequence from human CD8 α (SEQ ID NO.1.) or a leader sequence having at least 95% identity with SEQ ID NO.1, preferably of SEQ ID NO. 1

In another embodiment, primary immune T cells of the invention express an EGFRVIII CAR comprising a leader sequence of SEQ ID NO.2 or a leader sequence having at least 95% identity with SEQ ID NO.2.

In one embodiment primary immune T cells of the invention express an EGFRVIII CAR comprising:
a binding domain specific for EGFRVIII, preferably a domain specific for human EGFRVIII, more preferably said domain specific for human EGFRVIII is a single-chain variable fragment (scFv),
a hinge from human CD8 alpha chain (from CD8 α)
a transmembrane domain from human CD8alpha(α)
a co-stimulatory signal molecule from human 4-1 BB
an intracellular signaling domain comprising a human CD3zeta signaling domain.

In one embodiment primary immune T cells of the invention express an EGFRVIII CAR comprising:
a binding domain specific for EGFRVIII, preferably a domain specific for human EGFRVIII, more preferably said domain specific for human EGFRVIII is a single-chain variable fragment (scFv).
a hinge from human IgG1
a transmembrane domain from human CD8alpha(α)
a co-stimulatory signal molecule from human 4-1 BB
an intracellular signaling domain comprising a human CD3zeta signaling domain.

The present invention encompasses primary immune T cells of the invention expressing an EGFRVIII CAR comprising a signal peptide of SEQ ID NO 1 or of SEQ ID NO 2.

The present invention provides primary immune T cells of the invention expressing an EGFRVIII CAR comprising anti-EGFRVII a scfv linked to a hinge, preferably a hinge from CD8α, IgG1 or FCRIII (See FIG. 2), more preferably a hinge from CD8α, even more preferably a hinge with a SEQ ID NO.4.

Preferably, the present invention provides primary immune T cells expressing an EGFRVIII CAR comprising:
a signal peptide, preferably a signal peptide from CD8alpha, more preferably a signal peptide from CD8alpha of SEQ ID NO. 1 or of SEQ ID NO. 2.
a (scFv) comprising a VH domain separated to a VL domain by a linker, said VH and VL contributing to the binding to EGFRVIII,
a hinge from human CD8 alpha chain or a Hinge from human IgG1
a transmembrane domain (TM) from CD8alpha(α)
a co-stimulatory signal molecule from human 4-1 BB
an intracellular signaling domain comprising the CD3zeta signaling domain.

More preferably, the present invention provides primary immune T cells expressing an EGFRVIII CAR comprising:
a signal peptide from human CD8alpha,
a (scFv) comprising a VH domain separated to a VL domain by a linker, said VH and VL contributing to the binding to EGFRVIII,
a hinge from human CD8 alpha chain or a Hinge from human IgG1
a transmembrane domain (TM) from CD8alpha(α)
a co-stimulatory signal molecule from human 4-1 BB
an intracellular signaling domain comprising the CD3zeta signaling domain.

Even more preferably, the present invention provides primary immune T cells expressing an EGFRVIII CAR comprising:
a signal peptide from human CD8alpha,
a (scFv) comprising a VH domain separated to a VL domain by a linker, said VH and VL contributing to the binding to EGFRVIII and said VH and VL domains being humanized
a hinge from human CD8 alpha chain or a Hinge from human IgG1
a transmembrane domain (TM) from human CD8alpha(α)
a co-stimulatory signal molecule from human 4-1 BB
an intracellular signaling domain comprising the human CD3zeta signaling domain.

The primary immune T cells expressing an EGFRVIII CAR of the invention comprise:
a leader sequence (for example a CD8 α leader sequence or a CD8α signal peptide)
an anti-EGFRVIII scfv comprising a VH, a linker, and a VL,
   a CD8 α hinge
   a CD8 α TM
   a co-stimulatory signal molecule from 4-1 BB
   an intracellular CD3zeta signaling domain.

The primary immune T cells expressing an EGFRVIII CAR of the invention comprise:
a leader sequence (for example a CD8 α leader sequence or a CD8α signal peptide)
an anti-EGFRVIII scfv comprising a VL, a linker, and a VH,
   a CD8 α hinge
   a CD8 α TM
   a co-stimulatory signal molecule from 4-1 BB
   an intracellular CD3zeta signaling domain.

The primary immune T cells expressing an EGFRVIII CAR of the invention consist in:
a leader sequence (for example a CD8 α leader sequence or a CD8α signal peptide)
an anti-EGFRVIII scfv comprising a VH, a linker, and a VL,
   a CD8 α hinge
   a CD8 α TM
   a co-stimulatory signal molecule from 4-1 BB
   an intracellular CD3zeta signaling domain

In one embodiment, said linker is a linker of formula (G4S)n wherein n is 1 to 3; preferably n=3 and said sequence is (G4S)3, more preferably of SEQ ID NO. 10.

The primary immune T cells expressing an EGFRVIII CAR of the invention comprise:
a human CD8α leader sequence (CD8 α leader or CD8α signal peptide)
an anti-EGFRVIII scfv comprising a VH, a linker, and a VL, advantageously the scfv is humanized,
   a human CD8 α hinge
   a human CD8 α TM
   a co-stimulatory signal molecule from 4-1 BB
   an intracellular CD3zeta signaling domain.

In one embodiment the present invention provides:

primary immune T cells expressing an EGFRVIII CAR of the invention comprising:
a human CD8α leader sequence (CD8 α leader or CD8α signal peptide) of SEQ ID NO. 1
an anti-EGFRVIII scfv comprising a VH of SEQ ID NO. 11, a linker of SEQ ID No 10, and a VL of SEQ ID NO 12 or a VH of SEQ ID NO. 13, a linker of SEQ ID No 10 and a VL of SEQ ID NO 14, advantageously the scfv is humanized,
   a human CD8 α hinge of SEQ ID NO.4,
   a human CD8 α TM of SEQ ID NO.6
   a co-stimulatory signal molecule from 4-1BB of SEQ ID NO.8
   an intracellular CD3zeta signaling domain of SEQ ID NO. 9.

In one embodiment, the present invention provides primary immune T cells expressing an EGFRVIII CAR of the invention comprising:
a human CD8α leader sequence (CD8 α leader or CD8α signal peptide) of SEQ ID NO. 1
   an anti-EGFRVIII scfv comprising a VL of SEQ ID NO 12, a linker of SEQ ID No 10, and a VH of SEQ ID NO. 11, a VL of SEQ ID NO 14, a linker of SEQ ID No 10 and a VH of SEQ ID NO. 13, advantageously the scfv is humanized.
a human CD8 α hinge of SEQ ID NO.4,
a human CD8 α TM of SEQ ID NO.6
a co-stimulatory signal molecule from 4-1BB of SEQ ID NO.8
an intracellular CD3zeta signaling domain of SEQ ID NO. 9.

In one embodiment, the present invention provides primary immune T cells expressing an EGFRVIII CAR of the invention comprising:
a signal peptide having an amino acid sequence with at least 80%, more preferably at least 90%, 95% 97%, 99% or 100% sequence identity with the polypeptide of SEQ ID NO. 1 or 2; preferably the signal peptide has an amino acid sequence with at least 80%, more preferably at least 90%, 95% 97%, 99% or 100% sequence identity with the polypeptide of SEQ ID NO 1.
a VH domain separated to a VL domain by a linker, said VH and VL contributing to the binding to EGFRVIII; said linker having at least 90%, 95% 97%, 99% or 100% sequence identity with the polypeptide of SEQ ID NO 10.
   Said VH domain having at least 90%, 95% 97%, 99% or 100% sequence identity with the polypeptide of SEQ ID NO 11 or SEQ ID NO 13
   Said VL domain having at least 90%, 95% 97%, 99% or 100% sequence identity with the polypeptide of SEQ ID NO 12 or SEQ ID NO 14.
a hinge derived from human CD8 alpha chain having an amino acid sequence with at least 80%, more preferably at least 90%, 95% 97%, 99% or 100% sequence identity with the polypeptide of SEQ ID NO. 4;
a transmembrane domain derived from CD8alpha(α) having an amino acid sequence with at least 90%, 91%, 92%, 93%, 94%, 95%, 96%, 97%, 98%, 99% or 100% identity with the polypeptide of SEQ ID NO. 6;
a co-stimulatory signal molecule derived from human 4-1 BB (or 4-1 BB intracellular domain) having an amino acid sequence with at least 70%, preferably at least 80%, more preferably at least 90%, 95% 97%, 99% or 100% sequence identity with amino acid sequence selected from the group consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9.

In a preferred embodiment, EGFRVIII CARs of the invention expressed in primary immune T cells of the invention do not comprise any sequence from CD28 or from human CD28, in particular from human CD28 intra signaling domain. In a more preferred embodiment, the EGFRVIII CARs of the present invention expressed in primary immune T cells of the invention do not comprise any sequence from human CD28, in particular from human CD28 intra signaling domain and further contains a signal peptide from CD8α, preferably fused to the VH domain of a scfv specific for EGFRVIII.

In one embodiment, the present invention provides primary immune T cells expressing an EGFRVIII CAR of SEQ ID NO. 24.

In one embodiment the present invention provides primary immune T cells expressing an EGFRVIII CAR of SEQ ID NO. 25.

In one embodiment the present invention provides primary immune T cells expressing an EGFRVIII CAR of SEQ ID NO. 26.

In one embodiment the present invention provides primary immune T cells expressing an EGFRVIII CAR of SEQ ID NO. 27.

In a preferred embodiment the present invention provides primary immune T cells expressing an EGFRVIII CAR of SEQ ID NO. 24 or of SEQ ID NO. 25, more preferably of SEQ ID NO. 24.

In a more preferred embodiment the present invention provides primary immune T cells expressing an EGFRVIII CAR of SEQ ID NO. 24 or of SEQ ID NO. 25, more preferably of SEQ ID NO. 24 exhibiting a CTL and/or degranulating activity towards an EGFRVIII-expressing cell, preferably towards an EGFRVIII-expressing cancer cell,

In one embodiment, the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 24.

In one embodiment the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 25.

In one embodiment the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 26.

In one embodiment the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 27.

The present invention also relates to isolated cells or cell lines susceptible to be obtained by said method to engineer cells. In particular said isolated cell comprises at least one CAR of the invention as described above. In another embodiment, said isolated cell comprises a population of CARs each one comprising different extracellular ligand binding domains. In particular, said isolated cell comprises exogenous polynucleotide sequence encoding CAR. Genetically modified immune cells of the present invention are activated and proliferate independently of antigen binding mechanisms.

In the scope of the present invention is also encompassed an isolated immune cell, preferably a T-cell obtained according to any one of the methods previously described. Said immune cell refers to a cell of hematopoietic origin functionally involved in the initiation and/or execution of innate and/or adaptative immune response. Said immune cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+ T-lymphocytes and CD8+ T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T-cell according to the method previously described. Modified cells resistant to an immunosuppressive treatment and susceptible to be obtained by the previous method are encompassed in the scope of the present invention.

As a preferred embodiment, the present invention provides T-cells or a population of T-cells endowed with an EGFRvIII CAR as described above, that do not express functional TCR and that a reactive towards EGFRvIII positive cells, for their allogeneic transplantation into patients.

Activation and Expansion of T Cells

Whether prior to or after genetic modification of the T cells, even if the genetically modified immune cells of the present invention are activated and proliferate independently of antigen binding mechanisms, the immune cells, particularly T-cells of the present invention can be further activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

Generally, the T cells of the invention are expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% 002). T cells that have been exposed to varied stimulation times may exhibit different characteristics.

In another particular embodiment, said cells can be expanded by co-culturing with tissue or cells. Said cells can also be expanded in vivo, for example in the subject's blood after administrating said cell into the subject.

Therapeutic Applications

The present invention provides a composition comprising a primary T cell expressing an EGFRVIII CAR of the invention and a pharmaceutically acceptable vehicle is another object of the present invention.

Also disclosed herein are primary T cells expressing an EGFRVIII CAR of the invention as a medicament, In particular for immunotherapy.

Also disclosed herein are primary T cells expressing an EGFRVIII CAR of the invention for use in the treatment of cancer or to attenuate inflammation.

In another embodiment, isolated cell obtained by the different methods or cell line derived from said isolated cell as previously described can be used as a medicament. In another embodiment, said medicament can be used for treating cancer, particularly for the treatment of B-cell lymphomas and leukemia in a patient in need thereof. In another embodiment, said isolated cell according to the invention or cell line derived from said isolated cell can be used in the manufacture of a medicament for treatment of a cancer in a patient in need thereof.

The term “cancer” refers to a disease characterized by the rapid and uncontrolled growth of one or several types of cells. Examples of cancers are described herein and, include but are not limited to, glioblastoma, breast cancer, prostate cancer, ovarian cancer, cervical cancer, skin cancer, pancreatic cancer, colorectal cancer, renal cancer, liver cancer, brain cancer, lymphoma, leukemia, lung cancer. Preferably, cancer prevented or treated with the EGFRvIII CAR of the invention is a glioma, preferably a glioblastoma, more preferably multiple glioblastoma.

The term “disease associated with expression of EGFRvIII” as used herein includes, but is not limited to, a disease associated with expression of EGFRvIII or condition linked to the activity of cells which express EGFRvIII including, tumor cells of various cancers such as, e.g., glioblastoma (including glioblastoma stem cells); breast, ovarian, and non-small cell lung carcinomas; head and neck squamous cell carcinoma; medulloblastoma, colorectal cancer, prostate cancer, and bladder carcinoma. Lyse is one of the mechanisms whereby the EGFRvIII CAR T cells of the invention acts against EGFRvIII-expressing cells, reducing or eliminating tumors, facilitating infiltration of immune cells of the hosts to the tumor site, and enhancing/extending anti-tumor responses.

In another aspect, the present invention relies on methods for treating patients in need thereof, said method comprising at least one of the following steps:
(a) providing an immune-cell obtainable by any one of the methods previously described;
(b) Administrating said transformed immune cells to said patient,

On one embodiment, said T cells of the invention can undergo robust in vivo T cell expansion and can persist for an extended amount of time.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

Cells that can be used with the disclosed methods are described in the previous section. Said treatment can be used to treat patients diagnosed wherein a pre-malignant or malignant cancer condition characterized by EGFRvIII-expressing cells, especially by an overabundance of EGFRvIII-expressing cells. Such conditions are found in cancers, such as lung cancer, anal cancers and glioblastoma multiforme.

Types of cancers to be treated with the CARs of the invention include, but are not limited lung cancer, anal cancers and glioblastoma multiforme. Adult tumors/cancers and pediatric tumors/cancers are also included.

The present invention provides compositions and methods for treating diseases and disorders associated with EGFRvIII. An example of a disease or disorder associated with EGFRvIII is glioma.

Glioma refers to a cancer of the central nervous system that begins in glial cells (e.g., cells that surround and support nerve cells and includes oligodendrocytes, astrocytes, microglia, and ependymal cells). Gliomas classified into more than seven types such as glioblastoma and anaplastic astrocytoma according to their detailed pathological tissue type.

Disease stage (tumor size, presence of distal metastasis) and histological malignancy are used when determining the degree of malignancy of primary brain tumors. Histological malignancy is classified into four levels, i.e., G to G4 according to the Guidelines for the Treatment of Brain Tumors ((2002) Kanehara & Co., Ltd.), and these correspond to WH 01 to WH04, respectively. The larger the number, the higher the degree of malignancy. For example, the malignancy of glioblastoma is G4 (WH04), while the malignancy of anaplastic astrocytoma is G3 (WH03), and both G3 and G4 are classified as malignant.

Thus, according to particular embodiments, the methods of this invention target malignant gliomas. In other aspects the invention targets glioblastoma multiforme (GBM) or multiple glioblastoma.

In further embodiments, the compositions and methods of the present invention may be used in the treatment of other gliomas including, but not limited to, anaplastic astrocytoma, giant cell glioblastoma, gliosarcoma, anaplastic oligodendroglioma, anaplastic ependymoma, choroid plexus carcinoma, anaplastic ganglioglioma, pineoblastoma, medulloepithelioma, ependymoblastoma, medulloblastoma, supratentorial primitive neuroectodermal tumor, and atypical teratoid/rhabdoid tumor.

Glioblastoma is the most common primary brain tumor in adults. More than half of the patients diagnosed with malignant primary brain tumors each year have glioblastoma multiforme. Glioblastoma multiforme is an anaplastic, highly cellular tumor, with high proliferation indices, microvascular proliferation and focal necrosis.

The highly proliferative nature of these lesions likely results from multiple mitogenic effects. One of the hallmarks of GBM is endothelial proliferation. A host of angiogenic growth factors and their receptors are found in GBMs.

Glioblastoma multiforme prognosis remains dismal. Survival time is less than 2 years for the majority of patients.

Primary glioblastoma multiforme develops de novo from glial cells, typically has a clinical history of less than six months, is more common in older patients and presents small-cell histology. Secondary glioblastoma multiforme develops over months or years from pre-existing low-grade astrocytomas, predominantly affects younger people and presents giant-cell histology.

Malignant gliomas are also known as high grade gliomas. They can affect the brain and the spinal cord. In some aspects, compositions and methods of the present invention may be used to treat subjects carrying a brain malignant glioma, for example, one that is chosen among anaplastic astrocytoma (AA), glioblastoma multiform (GBM), anaplastic oligodendroglioma (AO) and anaplastic oligoastrocytoma (AOA).

Compositions and methods of the present invention may be used to treat a subject who has been characterized as having cells or tissues expressing EGFRvIII, or is suspected of having cells or tissues expressing EGFRvIII. For example, subjects benefiting from treatment according to the invention include subjects with a glioma, or subjects suspected of having a glioma, for example, as evidenced by the presence of one or more of headaches, nausea and vomiting, seizures, loss of vision, pain, weakness, numbness in the extremities, and/or cranial nerve disorders as a result of increased intracranial pressure. In particular embodiments, the glioma being treated is glioblastoma multiforme. In accordance with this embodiment, the glioblastoma multiforme can be in the brain or spinal cord.

In the present invention, an immune cell means a primary immune cell, an isolated primary immune cell, an isolated primary immune T cell, an isolated primary immune NK cell, an isolated primary immune TCR-KO T cell, preferably an isolated primary immune TCR-KO T cell which is resistant to a chemotherapy, such as to a drug selected from a purine nucleotide analogue, platine (cisplatine or carboplatine), anti-topoisomerase I (Irinotecan), anti-topoisomerase II (Etoposide), Methotrexate (folic acid analogs),

Preferably, the present invention provides a primary T cell expressing an efficient EGFRVIII CAR of the invention for use in the treatment of glioblastoma, more particularly, multiple glioblastoma. More preferably, the present invention provides a primary T cell expressing an efficient EGFRVIII CAR of the invention of SEQ ID N.O. 24 optionally humanized for use in the treatment of glioblastoma, more particularly, glioblastoma multiform.

In one embodiment patients are Patients With Residual or recurrent EGFRvIII+ Glioma, residual or recurrent EGFRvIII+ Glioblastoma.

In another embodiment, the present invention provides a primary T cell expressing an efficient EGFRVIII CAR of the invention for use in the treatment of glioblastoma, more particularly, multiple glioblastoma. More preferably, the present invention provides a primary T cell expressing an efficient EGFRVIII CAR of the invention of SEQ ID N.O. 24, optionally humanized for use in the treatment of patients With anaplastic astrocytoma glioblastoma glioma gliosarcoma or neuroepithelioma.

Also disclosed herein are primary T cells expressing an EGFRVIII CAR of the invention exhibiting a CTL and/or degranulating activity towards an EGFRVIII-expressing cell, preferably towards an EGFRVIII-expressing cancer cell, for use in the treatment of cancer, preferably of Residual or recurrent EGFRvIII+ Glioma, more preferably of glioblastoma multiforme (GBM).

Is provided herein, primary immune T cells expressing an EGFRVIII CAR comprising:
a binding domain specific for EGFRVIII, preferably a binding domain specific for human EGFRVIII, more preferably said binding domain specific for human EGFRVIII is a single-chain variable fragment (scFv).
a hinge,
a transmembrane domain,
a co-stimulatory signal molecule from human 4-1 BB, and
   an intracellular signaling domain comprising a human CD3zeta signaling domain, for use in the treatment of cancer, preferably of Residual or Reccurent EGFRvIII+ Glioma, more preferably of glioblastoma multiforme (GBM).

In a preferred embodiment, the primary immune T cells of the invention express an EGFRVIII CAR comprising: no CD28-derived sequence, in particular have no sequence having at least 1, at least 2, at least 3, at least 4, at least 5, at least 6, at least 7 at least 8, at least 9 at least 10 amino acids identity with human CD28 and are provided for use in the treatment of cancer, preferably of Residual or Recurrent EGFRvIII+ Glioma, more preferably of glioblastoma multiforme (GBM).

In a more preferred embodiment, the intracytoplasmic and/or transmembrane domain of the EGFRVIII CAR of the invention in the primary immune T cells of the invention, does not include human CD28-derived sequence, in particular has no sequence having at least 1, at least 2 at least 3, at least 4 at least 5, at least 6, at least 7 at least 8, at least 9 at least 10 amino acids identity with human CD28 and is provided for use in the treatment of cancer, preferably of residual or recurrent EGFRvIII+ Glioma, more preferably of glioblastoma multiforme (GBM),

Primary immune T cells of the invention expressing an EGFRVIII CAR comprising:
a binding domain specific for EGFRVIII, preferably a binding domain specific for human EGFRVIII, more preferably said binding domain specific for human EGFRVIII is a single-chain variable fragment (scFv),
a hinge,
a transmembrane domain,
a co-stimulatory signal molecule from human 4-1 BB,
an intracellular signaling domain consisting in a human CD3zeta signaling domain and no human CD28 signaling domain, for use in the treatment of cancer, preferably of Residual or recurrent EGFRvIII+ Glioma, more preferably of glioblastoma multiforme (GBM), are provided.

In a preferred embodiment, primary immune T cells of the invention expressing an EGFRVIII CAR comprising: no sequence from CD28 and a signal peptide (leader sequence), a TM domain and a hinge from CD8 α for use in the treatment of cancer, preferably of Residual or recurrent EGFRvIII+ Glioma, more preferably of glioblastoma multiforme (GBM), are provided.

In one embodiment, primary immune T cells of the invention expressing an EGFRVIII CAR comprising a leader sequence from human CD8 α or a leader sequence having at least 95% identity with SEQ ID NO.1, preferably of SEQ ID NO. 1 for use in the treatment of cancer, preferably of residual or recurrent EGFRvIII+ Glioma, more preferably of glioblastoma multiforme (GBM), are provided.

In another embodiment, primary immune T cells of the invention expressing an EGFRVIII CAR comprising a leader sequence of SEQ ID NO.2 or a leader sequence having at least 95% identity with SEQ ID NO.2 for use in the treatment of cancer, preferably of Residual or recurrent EGFRvIII+ Glioma, more preferably of glioblastoma multiforme (GBM), are provided.

In one embodiment primary immune T cells of the invention expressing an EGFRVIII CAR comprising:
a binding domain specific for EGFRVIII, preferably a domain specific for human EGFRVIII, more preferably said domain specific for human EGFRVIII is a single-chain variable fragment (scFv),
a hinge from human CD8 alpha chain (from CD8 α)
a transmembrane domain from human CD8alpha(α)
a co-stimulatory signal molecule from human 4-1 BB
an intracellular signaling domain comprising a human CD3zeta signaling domain for use in the treatment of cancer, preferably of Residual or Recurrent EGFRvIII+ Glioma, more preferably of glioblastoma multiforme (GBM), are provided.

Primary immune T cells of the invention expressing an EGFRVIII CAR comprising:
a binding domain specific for EGFRVIII, preferably a domain specific for human EGFRVIII, more preferably said domain specific for human EGFRVIII is a single-chain variable fragment (scFv).
a hinge from human IgG1
a transmembrane domain from human CD8alpha(α)
a co-stimulatory signal molecule from human 4-1 BB
an intracellular signaling domain comprising a human CD3zeta signaling domain for use in the treatment of cancer, preferably of Residual or Recurrent EGFRvIII+ Glioma, more preferably of glioblastoma multiforme (GBM), are provided.

The present invention encompasses primary immune T cells expressing an EGFRVIII CAR comprising a signal peptide of SEQ ID NO 1 or of SEQ ID NO 2 for use in the treatment of cancer, preferably of Residual or Recurrent EGFRvIII+ Glioma, more preferably of glioblastoma multiforme (GBM).

The present invention provides primary immune T cells expressing an EGFRVIII CAR comprising anti-EGFRVII a scfv linked to a hinge preferably a hinge from CD8α, IgG1 or FCRIII (See FIG. 2), more preferably a hinge from CD8α, even more preferably a hinge with a SEQ ID NO.4, for use in the treatment of cancer, preferably of residual or recurrent EGFRvIII+ Glioma, more preferably of glioblastoma multiforme (GBM).

The present invention provides primary immune T cells expressing an EGFRVIII CAR comprising anti-EGFRVII a scfv linked to a hinge preferably a hinge from CD8α, a TM from CD8α, and a signal peptide from CD8α even more preferably a hinge with a SEQ ID NO.4 a TM from CD8α, and a signal peptide from CD8α, for use in the treatment of cancer, preferably of Residual or recurrent EGFRvIII+ Glioma, more preferably of glioblastoma multiforme (GBM).

Preferably, the present invention provides primary immune T cells expressing an EGFRVIII CAR comprising:
a signal peptide, preferably a signal peptide from CD8alpha, more preferably a signal peptide from CD8alpha of SEQ ID NO. 1.
a (scFv) comprising a VH domain separated to a VL domain by a linker, said VH and VL contributing to the binding to EGFRVIII,
a hinge from human CD8 alpha chain or a Hinge from human IgG1
a transmembrane domain (TM) from CD8alpha(α)
a co-stimulatory signal molecule from human 4-1 BB
an intracellular signaling domain comprising the CD3zeta signaling domain for use in the treatment of cancer, preferably of Residual or recurrent EGFRvIII+ Glioma, more preferably of glioblastoma multiforme.

More preferably, the present invention provides primary immune T cells expressing an EGFRVIII CAR comprising:
a signal peptide from human CD8alpha,
a (scFv) comprising a VH domain separated to a VL domain by a linker, said VH and VL contributing to the binding to EGFRVIII,
a hinge from human CD8 alpha chain or a Hinge from human IgG1
a transmembrane domain (TM) from CD8alpha(α)
a co-stimulatory signal molecule from human 4-1 BB
an intracellular signaling domain comprising the CD3zeta signaling domain for use in the treatment of cancer, preferably of Residual or recurrent EGFRvIII+ Glioma, more preferably of glioblastoma multiforme (GBM).

Even more preferably, the present invention provides primary immune T cells expressing an EGFRVIII CAR comprising:
a signal peptide from human CD8alpha,
a (scFv) comprising a VH domain separated to a VL domain by a linker, said VH and VL contributing to the binding to EGFRVIII and said VH and VL domains being humanized
a hinge from human CD8 alpha chain or a Hinge from human IgG1
a transmembrane domain (TM) from human CD8alpha(α)
a co-stimulatory signal molecule from human 4-1 BB
an intracellular signaling domain comprising the human CD3zeta signaling domain for use in the treatment of cancer, preferably of residual or recurrent EGFRvIII+ Glioma, more preferably of glioblastoma multiforme (GBM), are provided.

A primary immune T cells expressing an EGFRVIII CAR comprising:
a leader sequence (for example a CD8 α leader sequence or a CD8α signal peptide)
an anti-EGFRVIII scfv comprising a VH, a linker, and a VL,
   a CD8 α hinge
   a CD8 α TM
   a co-stimulatory signal molecule from 4-1 BB
   an intracellular CD3zeta signaling domain for use in the treatment of cancer, preferably of Residual or recurrent EGFRvIII+ Glioma, more preferably of glioblastoma multiforme (GBM), are provided.
   A primary immune T cells expressing an EGFRVIII CAR comprising:
a leader sequence (for example a CD8 α leader sequence or a CD8α signal peptide)
an anti-EGFRVIII scfv comprising a VL, a linker, and a VH,
   a CD8 α hinge
   a CD8 α TM
   a co-stimulatory signal molecule from 4-1 BB
   an intracellular CD3zeta signaling domain, for use in the treatment of cancer, preferably of residual or recurrent EGFRvIII+ Glioma, more preferably of glioblastoma multiforme (GBM), are provided.

The primary immune T cells expressing an EGFRVIII CAR of the invention consisting in:
a leader sequence (for example a CD8 α leader sequence or a CD8α signal peptide)
an anti-EGFRVIII scfv comprising a VH, a linker, and a VL,
   a CD8 α hinge
   a CD8 α TM
   a co-stimulatory signal molecule from 4-1 BB
   an intracellular CD3zeta signaling domain for use in the treatment of cancer, preferably of Residual or recurrent EGFRvIII+ Glioma, more preferably of glioblastoma multiforme (GBM), are provided.

In one embodiment, said linker is a linker of formula (G4S)n wherein n is 1 to 3; preferably n=3 and said sequence is (G4S)3, more preferably of SEQ ID NO. 10.

A primary immune T cells expressing an EGFRVIII CAR of the invention comprising:
a human CD8α leader sequence (CD8 α leader or CD8α signal peptide)
an anti-EGFRVIII scfv comprising a VH, a linker, and a VL, advantageously the scfv is humanized,
   a human CD8 α hinge
   a human CD8 α TM
   a co-stimulatory signal molecule from 4-1 BB
   an intracellular CD3zeta signaling domain for use in the treatment of cancer, preferably of residual or recurrent EGFRvIII+ Glioma, more preferably of glioblastoma multiforme (GBM), are provided.

In one embodiment the present invention provides:

primary immune T cells expressing an EGFRVIII CAR of the invention comprising:
a human CD8α leader sequence (CD8 α leader or CD8α signal peptide) of SEQ ID NO. 1
an anti-EGFRVIII scfv comprising a VH of SEQ ID NO. 11, a linker of SEQ ID No 10, and a VL of SEQ ID NO 12 or a VH of SEQ ID NO. 13, a linker of SEQ ID No 10 and a VL of SEQ ID NO 14, advantageously the scfv is humanized,
   a human CD8 α hinge of SEQ ID NO.4,
   a human CD8 α TM of SEQ ID NO.6
   a co-stimulatory signal molecule from 4-1BB of SEQ ID NO.8
   an intracellular CD3zeta signaling domain of SEQ ID NO. 9 for use in the treatment of cancer, preferably of Residual or Recurrent EGFRvIII+ Glioma, more preferably of glioblastoma multiforme (GBM).

In one embodiment, the present invention provides

primary immune T cells expressing an EGFRVIII CAR of the invention comprising:
a human CD8α leader sequence (CD8 α leader or CD8α signal peptide) of SEQ ID NO. 1 an anti-EGFRVIII scfv comprising a VL of SEQ ID NO 12, a linker of SEQ ID No 10, and a VH of SEQ ID NO. 11, a VL of SEQ ID NO 14, a linker of SEQ ID No 10 and a VH of SEQ ID NO. 13, advantageously the scfv is humanized.
   a human CD8 α hinge of SEQ ID NO.4,
   a human CD8 α TM of SEQ ID NO.6
   a co-stimulatory signal molecule from 4-1BB of SEQ ID NO.8
   an intracellular CD3zeta signaling domain of SEQ ID NO. 9 for use in the treatment of cancer, preferably of Residual or recurrent EGFRvIII+ Glioma, more preferably of glioblastoma multiforme (GBM).

In one embodiment, the present invention provides primary immune T cells expressing an EGFRVIII CAR of the invention comprising:
a signal peptide having an amino acid sequence with at least 80%, more preferably at least 90%, 95% 97%, 99% or 100% sequence identity with the polypeptide of SEQ ID NO. 1 or 2; preferably the signal peptide has an amino acid sequence with at least 80%, more preferably at least 90%, 95% 97%, 99% or 100% sequence identity with the polypeptide of SEQ ID NO 1.
a VH domain separated to a VL domain by a linker, said VH and VL contributing to the binding to EGFRVIII; said linker having at least 90%, 95% 97%, 99% or 100% sequence identity with the polypeptide of SEQ ID NO 10.
   Said VH domain having at least 90%, 95% 97%, 99% or 100% sequence identity with the polypeptide of SEQ ID NO 11 or SEQ ID NO 13
   Said VL domain having at least 90%, 95% 97%, 99% or 100% sequence identity with the polypeptide of SEQ ID NO 12 or SEQ ID NO 14.
a hinge derived from human CD8 alpha chain having an amino acid sequence with at least 80%, more preferably at least 90%, 95% 97%, 99% or 100% sequence identity with the polypeptide of SEQ ID NO. 4;
a transmembrane domain derived from CD8alpha(α) having an amino acid sequence with at least 90%, 91%, 92%, 93%, 94%, 95%, 96%, 97%, 98%, 99% or 100% identity with the polypeptide of SEQ ID NO. 6;
a co-stimulatory signal molecule derived from human 4-1 BB (or 4-1 BB intracellular domain) having an amino acid sequence with at least 70%, preferably at least 80%, more preferably at least 90%, 95% 97%, 99% or 100% sequence identity with amino acid sequence consisting of SEQ ID NO: 8;
an intracellular signaling domain comprising the CD3zeta signaling domain having an amino acid sequence with at least 70%, preferably at least 80%, more preferably at least 90%, 95% 97%, 99% or 100% sequence identity with amino acid sequence selected from the group consisting of SEQ ID NO: 9, for use in the treatment of cancer, preferably of residual or recurrent EGFRvIII+ Glioma, more preferably of glioblastoma multiforme (GBM).

In a preferred embodiment, EGFRVIII CARs of the invention expressed in primary immune T cells for use in the treatment of cancer, preferably of residual or recurrent EGFRvIII+ Glioma, more preferably of glioblastoma multiforme (GBM), do not comprise any sequence from CD28 or from human CD28, in particular from human CD28 intra signaling domain.

In a more preferred embodiment, the EGFRVIII CARs of the present invention expressed in primary immune T cells of the invention for use in the treatment of cancer, preferably of residual or recurrent EGFRvIII+ Glioma, more preferably of glioblastoma multiforme (GBM), do not comprise any sequence from human CD28, in particular from human CD28 intra signaling domain and further contains a signal peptide from CD8□, preferably fused to the VH domain of a scfv specific for EGFRVIII.

In one embodiment, the present invention provides primary immune T cells expressing an EGFRVIII CAR of SEQ ID NO. 24 for use in the treatment of cancer, preferably of residual or recurrent EGFRvIII+ Glioma, more preferably of glioblastoma multiforme (GBM).

In one embodiment the present invention provides primary immune T cells expressing an EGFRVIII CAR of SEQ ID NO. 25 for use in the treatment of cancer, preferably of residual or recurrent EGFRvIII+ Glioma, more preferably of glioblastoma multiforme (GBM).

In one embodiment the present invention provides primary immune T cells expressing an EGFRVIII CAR of SEQ ID NO. 26 for use in the treatment of cancer, preferably of residual or recurrent EGFRvIII+ Glioma, more preferably of glioblastoma multiforme (GBM).

In one embodiment the present invention provides primary immune T cells expressing an EGFRVIII CAR of SEQ ID NO. 27 for use in the treatment of cancer, preferably of residual or recurrent EGFRvIII+ Glioma, more preferably of glioblastoma multiforme (GBM).

In a preferred embodiment the present invention provides primary immune T cells expressing an EGFRVIII CAR of SEQ ID NO. 24 or of SEQ ID NO. 25 for use in the treatment of cancer, preferably of residual or recurrent EGFRvIII+ Glioma, more preferably of glioblastoma multiforme (GBM), more preferably of SEQ ID NO. 24. for use in the treatment of cancer, preferably of residual or recurrent EGFRvIII+ Glioma, more preferably of glioblastoma multiforme (GBM).

In a more preferred embodiment the present invention provides primary immune T cells expressing an EGFRVIII CAR of SEQ ID NO. 24 or of SEQ ID NO. 25 for use in the treatment of cancer, preferably of residual or recurrent EGFRvIII+ Glioma, more preferably of glioblastoma multiforme (GBM), more preferably of SEQ ID NO. 24 exhibiting a CTL and/or degranulating activity towards an EGFRVIII-expressing cell, preferably towards an EGFRVIII-expressing cancer cell, for use in the treatment of cancer, preferably of Residual or recurrent EGFRvIII+ Glioma, more preferably of glioblastoma multiforme (GBM).

In one embodiment, the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 24 for use in the treatment of cancer, preferably of residual or recurrent EGFRvIII+ Glioma, more preferably of glioblastoma multiforme (GBM).

In one embodiment the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 25 for use in the treatment of cancer, preferably of residual or recurrent EGFRvIII+ Glioma, more preferably of glioblastoma multiforme (GBM).

In one embodiment the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 26 for use in the treatment of cancer, preferably of residual or recurrent EGFRvIII+ Glioma, more preferably of glioblastoma multiforme (GBM).

In one embodiment the present invention provides primary immune T cells expressing an EGFRVIII CAR having an amino acid sequence with at least 90%, 91%, 92%, 93%, 94%, 95%, 96%, 97%, 98%, 99% or 100% identity with the polypeptide of SEQ ID No 27 for use in the treatment of cancer, preferably of residual or recurrent EGFRvIII+ Glioma, more preferably of glioblastoma multiforme (GBM).

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993).

In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

### Other Definitions

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
By chimeric antigen receptor (CAR) is intended molecules that combine a binding domain against a component present on the target cell, for example an antibody-based specificity for a desired antigen (e.g., tumor antigen) with a T cell receptor-activating intracellular domain to generate a chimeric protein that exhibits a specific anti-target cellular immune activity. Generally, CAR consists of an extracellular single chain antibody (scFvFc) fused to the intracellular signaling domain of the T cell antigen receptor complex zeta chain (scFvFc:ζ) and have the ability, when expressed in T cells, to redirect antigen recognition based on the monoclonal antibody's specificity. One example of CAR used in the present invention is a CAR directing against EGFRvIII antigen and can comprise as non-limiting example the amino acid sequences: SEQ ID NO: 15 to 18, preferably SEQ ID NO.24 to 27, more preferably SEQ ID NO.24; more preferably humanized SEQ ID NO.24 to 27, more preferably humanized SEQ ID NO.24;

By primary T cell expressing a CAR of the invention, is intended primary T cell expressing molecules that combine at least one binding domain against EGFRvIII, for example an antibody-based specificity for a desired tumor antigen (EGFRvIII), with a T cell receptor-activating intracellular domain, to generate a chimeric protein that exhibits a specific anti-target cellular immune activity (CTL activity).

Generally, T cells expressing an EGFRvIII,CAR of the invention redirect antigen recognition based on the monoclonal antibody's specificity and induce the destruction of targeted cells. —The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 12 base pairs (bp) in length, more preferably of 14-55 bp. Rare-cutting endonucleases significantly increase HR by inducing DNA double-strand breaks (DSBs) at a defined locus (Perrin, Buckle et al. 1993; Rouet, Smih et al. 1994; Choulika, Perrin et al. 1995; Pingoud and Silva 2007). Rare-cutting endonucleases can for example be a homing endonuclease (Paques and Duchateau 2007), a chimeric Zinc-Finger nuclease (ZFN) resulting from the fusion of engineered zinc-finger domains with the catalytic domain of a restriction enzyme such as Fok I (Porteus and Carroll 2005), a Cas9 endonuclease from CRISPR system (Gasiunas, Barrangou et al. 2012; Jinek, Chylinski et al. 2012; Cong, Ran et al. 2013; Mali, Yang et al. 2013) or a chemical endonuclease (Eisenschmidt, Lanio et al. 2005; Arimondo, Thomas et al. 2006). In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer 2005). Such chemical endonucleases are comprised in the term “endonuclease” according to the present invention.
By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-Tevl, ColE7, NucA and Fok-I. In a particular embodiment, the TALE domain can be fused to a meganuclease like for instance I-Crel and I-Onul or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-Tevl described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALE-nuclease have been already described and used to stimulate gene targeting and gene modifications (Boch, Scholze et al. 2009; Moscou and Bogdanove 2009; Christian, Cermak et al. 2010; Li, Huang et al. 2011). Custom-made TAL-nucleases are commercially available under the trade name TALEN™ (Cellectis, 8 rue de la Croix Jerry, 75013 Paris, France).

The rare-cutting endonuclease according to the present invention can also be a Cas9 endonuclease. Recently, a new genome engineering tool has been developed based on the RNA-guided Cas9 nuclease (Gasiunas, Barrangou et al. 2012; Jinek, Chylinski et al. 2012; Cong, Ran et al. 2013; Mali, Yang et al. 2013) from the type II prokaryotic CRISPR (Clustered Regularly Interspaced Short palindromic Repeats) adaptive immune system (see for review (Sorek, Lawrence et al. 2013)). The CRISPR Associated (Cas) system was first discovered in bacteria and functions as a defense against foreign DNA, either viral or plasmid. CRISPR-mediated genome engineering first proceeds by the selection of target sequence often flanked by a short sequence motif, referred as the proto-spacer adjacent motif (PAM). Following target sequence selection, a specific crRNA, complementary to this target sequence is engineered. Trans-activating crRNA (tracrRNA) required in the CRISPR type II systems paired to the crRNA and bound to the provided Cas9 protein. Cas9 acts as a molecular anchor facilitating the base pairing of tracRNA with cRNA (Deltcheva, Chylinski et al. 2011). In this ternary complex, the dual tracrRNA:crRNA structure acts as guide RNA that directs the endonuclease Cas9 to the cognate target sequence. Target recognition by the Cas9-tracrRNA:crRNA complex is initiated by scanning the target sequence for homology between the target sequence and the crRNA. In addition to the target sequence-crRNA complementarity, DNA targeting requires the presence of a short motif adjacent to the protospacer (protospacer adjacent motif—PAM). Following pairing between the dual-RNA and the target sequence, Cas9 subsequently introduces a blunt double strand break 3 bases upstream of the PAM motif (Garneau, Dupuis et al. 2010).

Rare-cutting endonuclease can be a homing endonuclease, also known under the name of meganuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Preferred homing endonuclease according to the present invention can be an I-Cre/variant.
By “delivery vector” or “delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available, examples are in FIG. 4 and FIG. 5).

Viral vectors include retrovirus, adenovirus, parvovirus (e. g. adenoassociated viruses), coronavirus, negative strand RNA viruses such as orthomyxovirus (e.g., influenza virus), rhabdovirus (e.g., rabies and vesicular stomatitis virus), paramyxovirus (e. g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as nonlimiting example, that are able to integrate the genome of a target cell. At the opposite by “non-integrative lentiviral vectors (or NILV)” is meant efficient gene delivery vectors that do not integrate the genome of a target cell through the action of the virus integrase.
Delivery vectors and vectors can be associated or combined with any cellular permeabilization techniques such as sonoporation or electroporation or derivatives of these techniques.
By cell or cells is intended any eukaryotic living cells, primary cells and cell lines derived from these organisms for in vitro cultures.
By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines.

As non-limiting examples cell lines can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

All these cell lines can be modified by the method of the present invention to provide cell line models to produce, express, quantify, detect, study a gene or a protein of interest; these models can also be used to screen biologically active molecules of interest in research and production and various fields such as chemical, biofuels, therapeutics and agronomy as non-limiting examples.
by “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “variant(s)”, it is intended a repeat variant, a variant, a DNA binding variant, a TALE-nuclease variant, a polypeptide variant obtained by mutation or replacement of at least one residue in the amino acid sequence of the parent molecule.
by “functional variant” is intended a catalytically active mutant of a protein or a protein domain; such mutant may have the same activity compared to its parent protein or protein domain or additional properties, or higher or lower activity.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.
“signal-transducing domain” or “co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor.

A “co-stimulatory signal” as used herein refers to a signal, which in combination with primary signal, such as TCR/CD3 ligation, leads to T cell proliferation and/or upregulation or downregulation of key molecules.

The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. Thus examples of cell surface markers that may act as ligands include those associated with viral, bacterial and parasitic infections, autoimmune disease and cancer cells.

The term “subject” or “patient” as used herein includes all members of the animal kingdom including non-human primates and humans. In one embodiment patients are humans with a glioma preferably residual or recurrent EGFRvIII+ Glioma. Patient can benefit a treatment according to the invention.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

### General Method

### EGFRvIII Cars

EGFRvIII CARs were prepared using different scfv according to a method previously described in documents US2010/0105136 and US2010/0105136 A1 which are incorporated herein by reference in entirety.

### Screening and Selection of CAR

Primary T-Cell Cultures

T cells were purified from Buffy coat samples provided by EFS (Etablissement Français du Sang, Paris, France) using Ficoll gradient density medium. The PBMC layer was recovered and T cells were purified using a commercially available T-cell enrichment kit. Purified T cells were activated in X-Vivo™-15 medium (Lonza) supplemented with 20 ng/mL Human IL-2, 5% Human, and Dynabeads Human T activator CD3/CD28 at a bead:cell ratio 1:1 (Life Technologies).

CAR mRNA Transfection

Transfections of CAR mRNAs encoding the different CAR constructs were done at Day 4 or Day 11 after T-cell purification and activation. Cells were immediately diluted in X-Vivo™-15 media and incubated at 37° C. with 5% CO₂. IL-2 was added 2 h after electroporation at 20 ng/mL.

T-Cell Transduction with Recombinant Lentiviral Vectors Allowing the Expression of CAR

Transduction of T-cells with recombinant lentiviral vectors expression the CAR was carried out three days after T-cell purification/activation. Lentiviral vectors produced by Vectalys SA (Toulouse, France) by transfection of genomic and helper plasmids in HEK-293 cells may be used. Transductions were carried out at a multiplicity of infection of 5. CAR detection at the surface of T-cells is performed using a recombinant protein consisting on the extracellular domain of the human EGFRVIII protein fused together with a murine IgG1 Fc fragment (produced by LakePharma). Binding of this protein to the CAR molecule is detected with a PE-conjugated secondary antibody (Jackson lmmunoresearch) targeting the mouse Fc portion of the protein, and analyzed by flow cytometry.

Inactivation of Specific Gene(s) in Primary T Cells

Inactivation of specific gene(s) in primary T cells may be performed before or after CAR introduction into T cells.
At least one gene is inactivated, one, two or three genes may be inactivated in one step; In a preferred embodiment two genes are inactivated, preferably TCRalpha gene and a gene conferring resistance to a drug selected from purine nucleotide analogues, platines (cisplatine or carboplatine), anti-topoisomerase I (Irinotecan), anti-topoisomerase II (Etoposide), Methotrexate (folic acid analogs),

In general, heterodimeric nuclease, in particular TALE-Nuclease targeting two long sequences (called half targets) separated by a spacer within a target gene is designed and produced.

Each TALE-nuclease construct may be cloned in an appropriate mammalian expression vector. mRNA encoding TALE-nuclease cleaving a targeted genomic sequence may be synthesized from plasmid carrying the coding sequence downstream a promoter. Purified T cells preactivated with anti-CD3/CD28 coated beads are used and transfected with each of the 2 mRNAs encoding both half TALE-nucleases. Cells may be reactivated with soluble anti-CD28 to measure cell proliferation for various times and the activation marker CD25 detected to assess the activation state of the cells.

Degranulation Assay (CD107a Mobilization)

T-cells were incubated in 96-well plates, together with an equal amount of cells expressing various levels of the targeted protein (EGFRVIII). Co-cultures were maintained for 6 hours at 37° C. with 5% CO₂. CD107a staining was done during cell stimulation, by the addition of a fluorescent anti-CD107a antibody at the beginning of the co-culture, together with an anti-CD49d, anti-CD28, and 1× Monensin solution, as a control. After the 6 h incubation period, cells were stained with a fixable viability dye and fluorochrome-conjugated anti-CD8 and analyzed by flow cytometry. The degranulation activity was determined as the % of CD8+/CD107a+ cells, and by determining the mean fluorescence intensity signal (MFI) for CD107a staining among CD8+ cells. Degranulation assays were carried out 24 h after mRNA transfection.

IFN Gamma Release Assay

24 h after mRNA transfection, CAR expressing T-cells were incubated together with cell lines expressing various levels of the targeted protein for 24 hours at 37° C. The supernatants were recovered and IFN gamma detection in the cell culture supernatants was done by ELISA assay.

Cytotoxicity Assay

T-cells were incubated together with 10,000 target cells (expressing various levels of the targeted protein) or (negative control) cells in the same well. Target and control cells were labelled with fluorescent intracellular dyes (CFSE or Cell Trace Violet) before co-culturing them with CAR+ T-cells. The co-cultures were incubated for 4 hours at 37° C. After this incubation period, cells were labelled with a fixable viability dye and analyzed by flow cytometry. Viability of each cellular population (target cells or negative control cells) was determined and the % of specific cell lysis was calculated. Cytotoxicity assays were carried out 48 h after mRNA transfection.

Anti-Tumor Mouse Model

Immuno deficient mice are implanted with tumor cells (glioblastoma) or with targeted protein expressing-Luciferase cells into the flank. Subsequently, cells were implanted into mouse brains. Serial transplantation into further generations of mice continues the maintenance of in vivo xenograft cell lines. Optionally, mice received an anti-cancer treatment before/or together with injection with CAR+ T-cells. Mice are then iv injected (either 2 or 7 days after injection of the tumor cell line) with different doses of CAR+ T-cells to be tested, or with T-cells that were not transduced with the CAR lentiviral vector. Bioluminescent signals are determined at the day of T-cell injection (D0), at D7, 14, 21, 28 and 40 after T-cell injection in order to follow tumoral progression in the different animals.

Chen, Jian et al. Any other model of glioma such as those described in Malignant Glioma: Lessons from Genomics, Mouse Models, and Stem Cells. Cell: Volume 149, Issue 1, 36-47 are suitable for the present study.

### Clinical Study

### Primary Objectives

To evaluate the safety and efficiency of the administration of TCR KO, primary T cell expressing an anti-EGFRvIII CAR (anti-EGFRvIII CAR-engineered T lymphocytes) in patients with brain cancer, glioblastoma or gliosarcoma in particular Glioblastoma, more particularly multiple Glioblastoma.

To determine the six month progression free survival of patients receiving anti-EGFRvIII CAR-engineered T lymphocytes and optionally aldesleukin following a nonmyeloablative but lymphoid depleting preparative regimen.

### Secondary Objectives

Determine the in vivo survival of anti-EGFRvIII CAR-engineered T lymphocytes.
Evaluate radiographic changes after treatment

### Eligibility:

Histologically proven glioblastoma or gliosarcoma expressing EGFRvIII as determined by IHC or RT-PCR
Failed prior standard treatment with radiotherapy with or without chemotherapy
Karnofsky score greater than or equal to 60%
Cardiac, pulmonary and laboratory parameters within acceptable limits.

### Design:

The study was conducted using a Phase I/II design.
Patients received a non-myeloablative but lymphocyte depleting preparative regimen consisting of cyclophosphamide and fludarabine followed by intravenous infusion of ex vivo tumor reactive, anti-EGFRvIII CAR-engineered T lymphocytes, optionally plus IV aldesleukin.
Once the MTD has been determined, the study proceeded to the phase II portion.
In the phase 2 portion of the trial, patients were accrued to two groups:
   Patients with recurrent malignant glioma requiring steroid use at the start of treatment
   Patients with recurrent malignant glioma not requiring steroids at the start of treatment

### Study Type Interventional

### Study Phase Phase 1-Phase 2

Study Design
Allocation: Non-Randomized
Endpoint Classification: Safety/Efficacy Study
Intervention Model: Single Group Assignment
Masking: Open Label
Primary Purpose: Treatment

### Condition

Glioma (Malignant)
Glioblastoma, multiple Glioblastoma
Brain Cancer

### Intervention

Biological: anti-EGFRvIII CAR-engineered T lymphocytes
On day 0 (one to four days after the last dose of fludarabine), cells were infused intravenously (i.v.) on the Patient Care Unit over 20 to 30 minutes.
Drug: None of vehicle
Drug: Aldesleukin
Aldesleukin (based on total body weight) 72,000 IU/kg IV over 15 minute approximately every eight hours (+/− one hour) beginning within 24 hours of cell infusion and continuing for up to 5 days maximum of 15 doses).
Drug: Fludarabine
Fludarabine 25 mg/m2/day IVPB daily over 30 minutes for 5 days.
Drug: Cyclophosphamide
   Cyclophosphamide 60 mg/kg/day×2 days IV in 250 ml D5W over 1 hr.

### Study Arm (s)

### Experimental: Single Arm—

Patients received a non-myeloablative but lymphocyte depleting preparative regimen consisting of cyclophosphamide and fludarabine followed by intravenous infusion of anti-EGFRvIII CAR-engineered T lymphocytes.

### Interventions:

Biological: anti-EGFRvIII CAR-engineered T lymphocytes
Drug: None
Drug: Aldesleukin as above
Drug: Fludarabine as above
Drug: Cyclophosphamide as above

Having generally described this invention, a further understanding can be obtained by reference to certain specific examples, which are provided herein for purposes of illustration only, and are not intended to be limiting unless otherwise specified.

### EXAMPLES

### Example 1: Proliferation of TCR Alpha Inactivated Cells Expressing an EGFRvIII-CAR

Heterodimeric TALE-nuclease targeting two 17-bp long sequences (called half targets) separated by an 15-bp spacer within T-cell receptor alpha constant chain region (TRAC) gene were designed and produced. Each half target is recognized by repeats of the half TALE-nucleases listed in Table 10.

TABLE 10
TAL-nucleases targeting
TCRalpha gene

| | | | Half |
|---|---|---|---|
| | Target | Repeat | TALE- |
| Target | sequence | sequence | nuclease |
| TRAC_T01 | TTGTCCCACAGATAT | Repeat | TRAC_T01-L |
| | CCAgaaccctgaccc | TRAC_T01-L | TALEN |
| | tgCCGTGTACCAGCT | (SEQ ID | (SEQ ID |
| | GAGA | NO: 20) | NO: 22) |
| | (SEQ ID NO: 19) | Repeat | TRAC_T01-R |
| | | TRAC_T01-R | TALEN |
| | | (SEQ ID | (SEQ ID |
| | | NO: 21) | NO: 23) |

Each TALE-nuclease construct was subcloned using restriction enzyme digestion in a mammalian expression vector under the control of the T7 promoter. mRNA encoding TALE-nuclease cleaving TRAC genomic sequence were synthesized from plasmid carrying the coding sequence downstream from the T7 promoter.

Purified T cells preactivated during 72 hours with anti-CD3/CD28 coated beads were transfected with each of the 2 mRNAs encoding both half TRAC_T01 TALE-nucleases. 48 hours post-transfection, different groups of T cells from the same donor were respectively transduced with a lentiviral vector encoding one of the EGFRvIII CAR previously described (SEQ ID NO: 15 to 18). 2 days post-transduction, CD3_{NEG} cells were purified using anti-CD3 magnetic beads and 5 days post-transduction cells were reactivated with soluble anti-CD28 (5 μg/ml).

Cell proliferation was followed for up to 30 days after reactivation by counting cell 2 times per week. Increased proliferation in TCR alpha inactivated cells expressing the EGFRvIII CARs, especially when reactivated with anti-CD28, was observed compared to non-transduced cells.

To investigate whether the human T cells expressing the EGFRvIII CAR display activated state, the expression of the activation marker CD25 are analyzed by FACS 7 days post transduction. The purified cells transduced with the lentiviral vector encoding EGFRvIII CAR assayed for CD25 expression at their surface in order to assess their activation in comparison with the non-transduced cells. Increased CD25 expression is expected both in CD28 reactivation or no reactivation conditions.

The present invention provides an engineered EGFRvIII CAR TCR KO T-cell targeting epidermal growth factor receptor variant III (EGFRvIII), for the treatment of glioblastoma.

The present invention provides an engineered EGFRvIII CAR TCR KO T-cell targeting epidermal growth factor receptor variant III (EGFRvIII), for the treatment of multiple Glioblastoma.

### Example 2: EGFRvIII CAR-T

Development of engineered CAR T-cells targeting epidermal growth factor receptor variant III (EGFRvIII), for the treatment of glioblastoma.

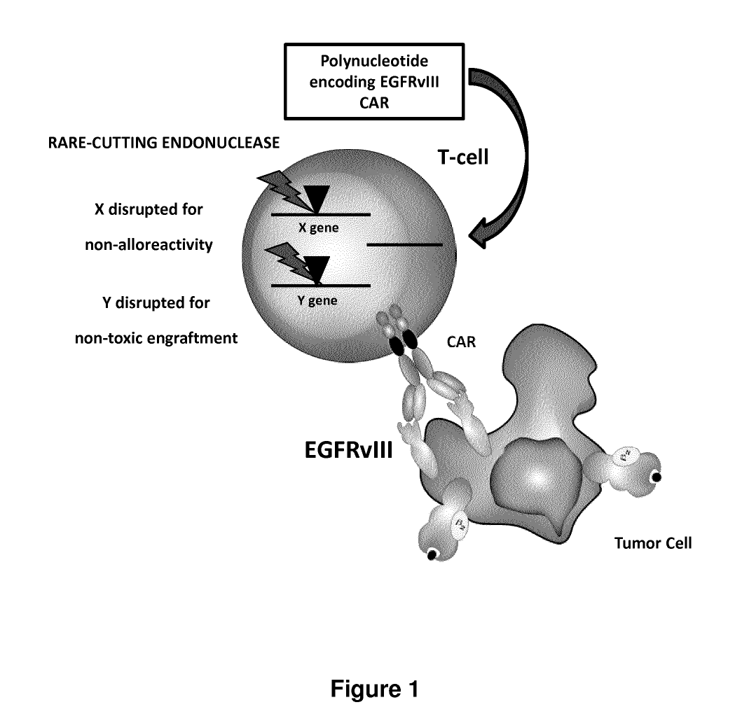

EGFRvIII is most common EGFR mutant and consists of an in-frame deletion of exons 2-7. This deletion results in a truncated extracellular ligand-binding domain, and renders the protein constitutively active in a ligand-independent fashion. EGFRvIII expression has been shown to enhance tumorigenicity, promote cellular motility, and confer resistance to radiation and chemotherapy. EGFRvIII expression has been reported in 24-67% of glioblastomas, but not in any normal tissues, making it an attractive target for immunotherapy with CAR T Cells (FIG. 1).

### 1. EGFRvIII Cars:

1.1. Construct

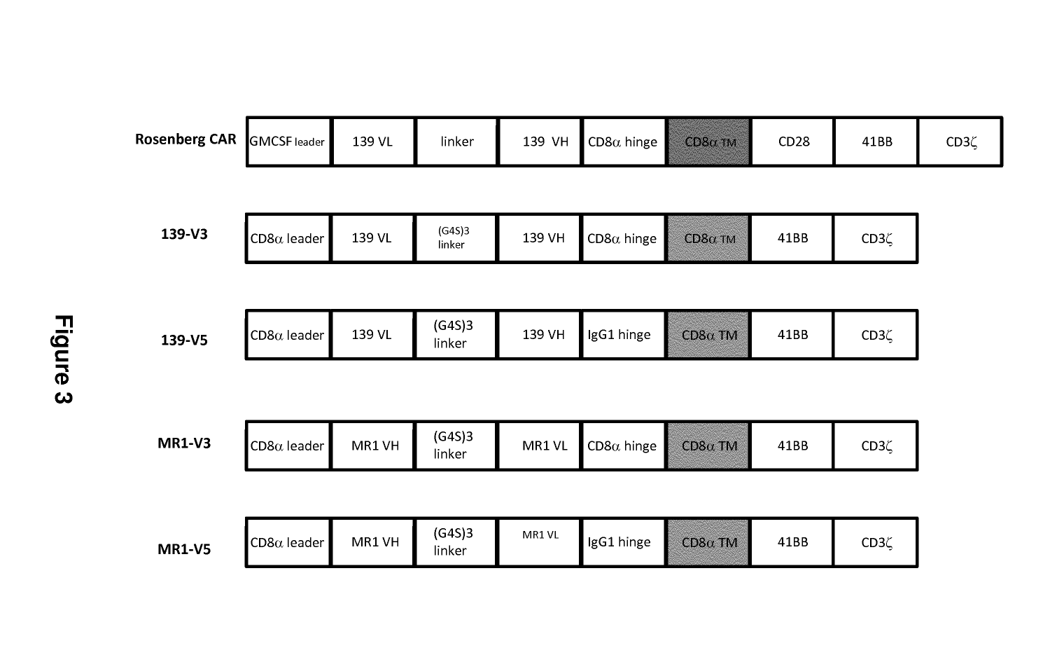

Four EGFRvIII CARs were designed (FIG. 2 and FIG. 3) and prepared using different scfv as previously described in documents US2010/0105136 and US2010/0105136 A1 which are incorporated herein by reference in entirety The 139 scfv derived from 139 antibody described by Rosenberg in a patent PCT/US2012/029861, or the MR1 scfv derived from MR1 antibody described by Carter in patent US2010/0105136 A1 were used. 2 different CARs architectures (FIG. 2 and FIG. 3) have been designed and prepared with the 41 BB costimulatory domain, the CD3ζ activation domain, the CD8α transmembrane domain and 2 different hinges, either a CD8α hinge (V3 architecture) or an IgG1 hinge (V5 architecture) (SEQ-ID No 24 to SEQ-ID No 27).

We also have produced the CAR described by Rosenberg to verify its activity (FIG. 3). Constructs were inserted into pCLS9632 (FIG. 4) for transient expression and screening of designed CARs.CAR constructs were introduced into this backbone by using AscI and HindIII restriction sites.

The same constructs were inserted into a pCL26700 psew EF1a BFP vector (pCL26700 vector) (FIG. 5) between XmaI and SpeI restriction sites for further transduction in primary T cells.

EGFRvIII CARs were 139-V3 CAR (SEQ ID NO.24) and the 139-V5 (SEQ ID NO.25) CAR, the MR1-V3 (SEQ ID NO.26) and the MR1-V5 CAR (SEQ ID NO.27).

Thus, present invention provides a pCL26700 psew EF1a BFP vector comprising a sequence coding an EGFRvIII CARs of the invention, such as a pCL26700 psew EF1a BFP vector comprising a sequence coding SEQ ID NO.24,

a pCL26700 psew EF1a BFP vector comprising a sequence coding SEQ ID NO.25,
a pCL26700 psew EF1a BFP vector comprising a sequence coding SEQ ID NO.26,
a pCL26700 psew EF1a BFP vector comprising a sequence coding SEQ ID NO.27, preferably, a pCL26700 psew EF1a BFP vector comprising a sequence coding SEQ ID NO.24,

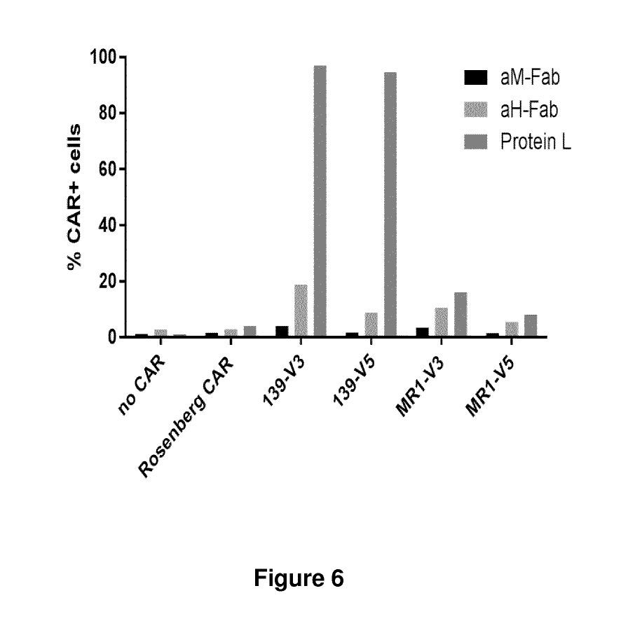

1.2. CAR Expression (FIG. 6)

CAR mRNAs were transfected into primary TCR KO T or T cells 5 days after activation by anti-CD3CD28 coated beads and IL-2. The CAR expression was assessed by flow cytometry. The 139-V3 CAR and the 139-V5 CAR were detected. The other CARs expression was low (MR1) or; undetectable (CAR designed by Rosenberg), by using this approach regardless of the architecture used (V3 or V5) (FIG. 6).

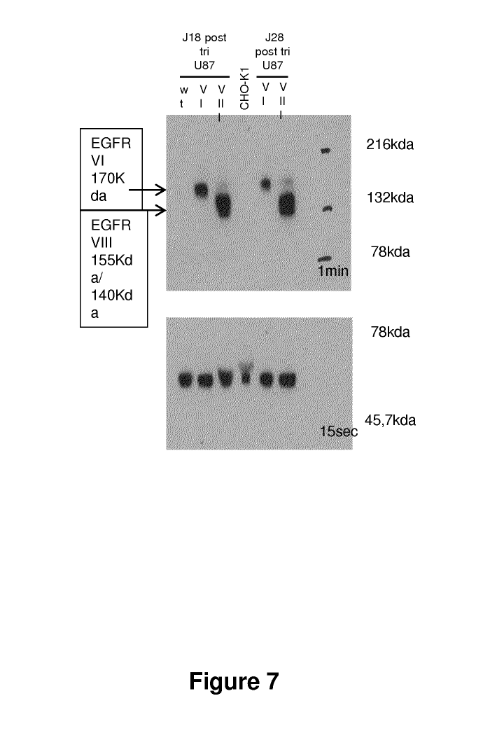

### 2. Production of EGFRvIII Cell Lines (FIG. 7)

To test the functionality of the anti-EGFRvIII CARs, U87 overexpressing EGFRvIII cell lines were produced.

2.1. Overexpression of EGFR and EGFRvIII in U87 Cells.

### 2.1.1. Cell Lines Development

U87 cells overexpressing EGFR (EGFRVI) or EGFRvIII were generated and characterized by FACS and Western-blot (FIG. 7). FIG. 7: shows U87 glioma cells overexpressing EGFRVI (170 Kda) or EGFRVIII (155 Kda/140 Kda) proteins.

The results obtained by FACS analysis showed that 56.6% of the cells express a detectable level of EGRFVI and 57.3% of the cells express a detectable level of EGRFVIII.

These EGFRVI expressing cells and EGFRVIII expressing cells were eventually sorted and isolated.

### 2.1.2. Validation of New Cell Lines as Target Cells for EGFRvIII CAR T Cells

2.1.2.1. Degranulation Assay

To validate the cell lines and the CAR constructs a degranulation assay has been performed on these new target cells with T cells expressing the EGFRvIII CARs (FIG. 3). The CART degranulation was evaluated by flow cytometry. The read-out is the CD107a expression at the T cell plasma membrane after 5 hours incubation with target cells. Surprisingly, the CARs 139-V3, CARs 139-V5, MR1-V3 and MR1-V5 were able to degranulate even when their expression was low. In contrast, the results showed that the Rosenberg CAR did not display activity in these experimental conditions.

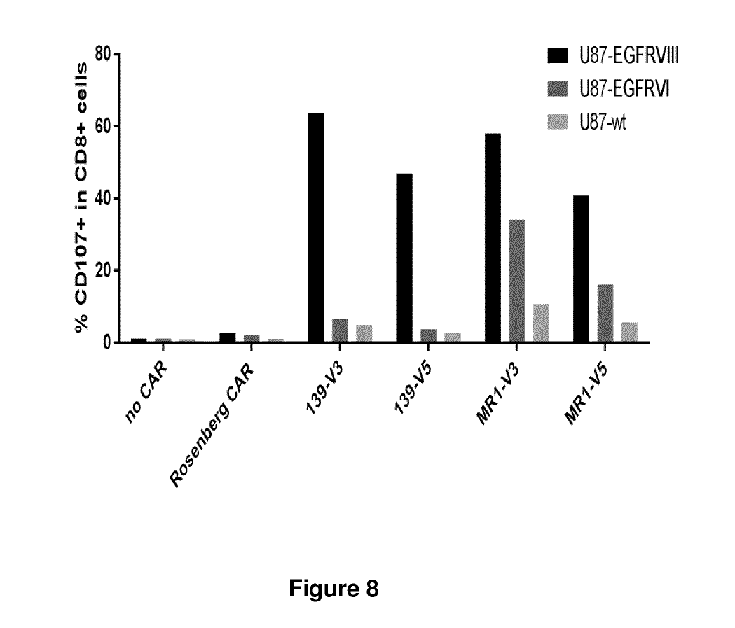

FIG. 8 shows EGFRvIII CART degranulation capacity assessed by FACS analysis after coculture with target cells. The Rosenberg CAR that was undetectable and did not degranulate and thus was not further studied.

2.1.2.2. Cytotoxicity Assay

A cytotoxicity assay has been performed on these new target cells with T cells expressing the 4 EGFRVIII CARs. EGFRvIII CARs of the invention, the results showed a specific lysis of EGFRvIII cells (U87-EGFRvIII) (FIG. 9) but neither on EGFRvI (U87-EGFR) cells nor U87 wt cells. FIG. 9 shows a cytotoxicity assay of EGFRvIII CART cells.

The data obtained show that EGFRvIII CARs T cells, of the invention especially those of V3 structure could significantly reduce glioma cells in vitro and in vivo, in colonized spinal cord and brain.

Examples of CAR polypeptide sequences:
Framed sequences correspond to preferred VH and VL sequences. VH
and VL may be swapped to improve CAR efficiency.
139-v1 (SEQ ID NO. 1 + SEQ ID NO. 15)

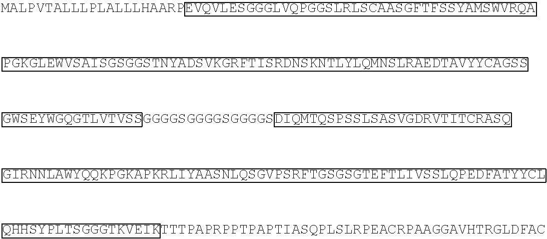

DIYIWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
139-v2 (SEQ ID NO. 1 + SEQ ID NO. 16)

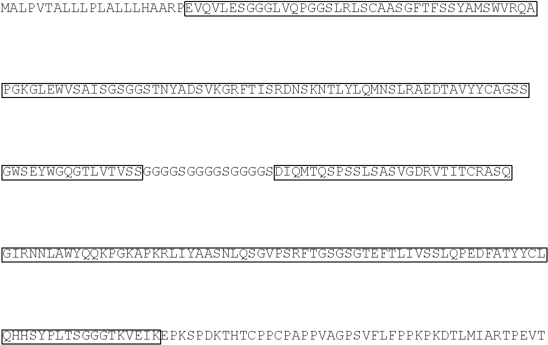

CVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSL
SLSPGKIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTYDALHMQALP
PR
MR1-v1 (SEQ ID NO. 1 + SEQ ID NO. 17)

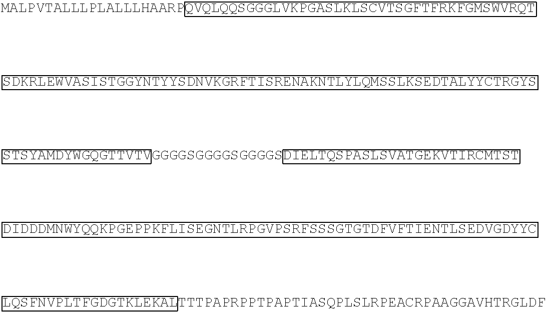

ACDIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
MR1-v2 (SEQ ID NO. 1 + SEQ ID NO. 18)

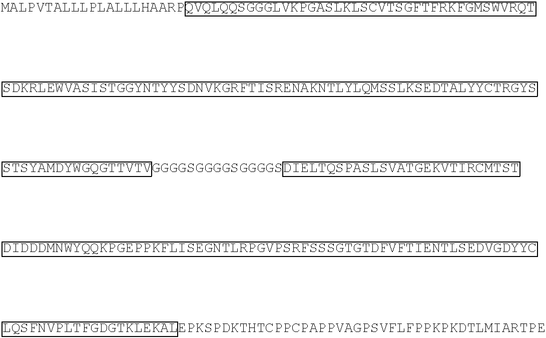

VTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQK
SLSLSPGKIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATKDTYDALHMQ
ALPPR
Sequences
SEQ-ID No 28: Rosenberg CAR
MVLLVTSLLLCELPHPAFLLIPDIQMTQSPSSLSASVGDRVTITCRASQGIRNNLAWYQQKPG
KAPKRLIYAASNLQSGVPSRFTGSGSGTEFTLIVSSLQPEDFATYYCLQHHSYPLTSGGGTK
VEIKRTGSTSGSGKPGSGEGSEVQVLESGGGLVQPGGSLRLSCAASGFTFSSYAMSWVR
QAPGKGLEWVSAISGSGGSTNYADSVKGRFTISRDNSKNTLYLQMNSLRAEDTAVYYCAG
SSGWSEYWGQGTLVTVSSAAAFVPVFLPAKPTTTPAPRPPTPAPTIASQPLSLRPEACRPA
AGGAVHTRGLDFACDIYIWAPLAGTCGVLLLSLVITLYCNHRNRSKRSRLLHSDYMNMTPRR
PGPTRKHYQPYAPPRDFAAYRSRFSVVKRGRKKLLYIFKQPFMRPVQTTQEEDGCSCRFP
EEEEGGCELRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTYDALHMQALP
PR*
SEQ-ID No 24: 139-v3
MALPVTALLLPLALLLHAARPDIQMTQSPSSLSASVGDRVTITCRASQGIRNNLAWYQQKPG
KAPKRLIYAASNLQSGVPSRFTGSGSGTEFTLIVSSLQPEDFATYYCLQHHSYPLTSGGGTK
VEIKGGGGSGGGGSGGGGSEVQVLESGGGLVQPGGSLRLSCAASGFTFSSYAMSWVRQ
APGKGLEWVSAISGSGGSTNYADSVKGRFTISRDNSKNTLYLQMNSLRAEDTAVYYCAGSS
GWSEYWGQGTLVTVSSTTTPAPRPPTPAPTIASQPLSLRPEACRPAAGGAVHTRGLDFAC
DIYIWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR*
SEQ-ID No 25: 139-v5
MALPVTALLLPLALLLHAARPDIQMTQSPSSLSASVGDRVTITCRASQGIRNNLAWYQQKPG
KAPKRLIYAASNLQSGVPSRFTGSGSGTEFTLIVSSLQPEDFATYYCLQHHSYPLTSGGGTK
VEIKGGGGSGGGGSGGGGSEVQVLESGGGLVQPGGSLRLSCAASGFTFSSYAMSWVRQ
APGKGLEWVSAISGSGGSTNYADSVKGRFTISRDNSKNTLYLQMNSLRAEDTAVYYCAGSS
GWSEYWGQGTLVTVSSEPKSPDKTHTCPPCPAPPVAGPSVFLFPPKPKDTLMIARTPEVTC
VVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLS
LSPGKKDPKIYIWAPLAGTCGVLLLSLVITLYCKRGRKKLLYIFKQPFMRPVQTTQEEDGCSC
RFPEEEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHM
QALPPR*
SEQ-ID No 26: MR1-v3
MALPVTALLLPLALLLHAARPQVQLQQSGGGLVKPGASLKLSCVTSGFTFRKFGMSWVRQT
SDKRLEWVASISTGGYNTYYSDNVKGRFTISRENAKNTLYLQMSSLKSEDTALYYCTRGYS
STSYAMDYWGQGTTVTVGGGGSGGGGSGGGGSDIELTQSPASLSVATGEKVTIRCMTST
DIDDDMNWYQQKPGEPPKFLISEGNTLRPGVPSRFSSSGTGTDFVFTIENTLSEDVGDYYC
LQSFNVPLTFGDGTKLEKALTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR*
SEQ-ID No 27: MR1-v5
MALPVTALLLPLALLLHAARPQVQLQQSGGGLVKPGASLKLSCVTSGFTFRKFGMSWVRQT
SDKRLEWVASISTGGYNTYYSDNVKGRFTISRENAKNTLYLQMSSLKSEDTALYYCTRGYS
STSYAMDYWGQGTTVTVGGGGSGGGGSGGGGSDIELTQSPASLSVATGEKVTIRCMTST
DIDDDMNWYQQKPGEPPKFLISEGNTLRPGVPSRFSSSGTGTDFVFTIENTLSEDVGDYYC
LQSFNVPLTFGDGTKLEKALEPKSPDKTHTCPPCPAPPVAGPSVFLFPPKPKDTLMIARTPE
VTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQK
SLSLSPGKKDPKIYIWAPLAGTCGVLLLSLVITLYCKRGRKKLLYIFKQPFMRPVQTTQEEDG
CSCRFPEEEEGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDA
LHMQALPPR*

### REFERENCES

Arimondo, P. B., C. J. Thomas, et al. (2006). “Exploring the cellular activity of camptothecin-triple-helix-forming oligonucleotide conjugates.” Mol Cell Biol 26(1): 324-33.
Atkins, J. F., N. M. Wills, et al. (2007). “A case for “StopGo”: reprogramming translation to augment codon meaning of GGN by promoting unconventional termination (Stop) after addition of glycine and then allowing continued translation (Go).” Rna 13(6): 803-10
Badhiwala J, Decker W K, Berens M E, Bhardwaj R D. Clinical trials in cellular immunotherapy for brain/CNS tumors. Expert Rev Neurother. 2013 April; 13(4):405-24. doi: 10.1586/ern.13.23. Review.
Bierer, B. E., G. Hollander, et al. (1993). “Cyclosporin A and FK506: molecular mechanisms of immunosuppression and probes for transplantation biology.” Curr Opin Immunol 5(5): 763-73.
Bloom H J. Combined modality therapy for intracranial tumors. Cancer. 1975 January; 35(1):111-20. Review.
Boch, J., H. Scholze, et al. (2009). “Breaking the code of DNA binding specificity of TAL-type III effectors.” Science 326(5959): 1509-12.
Choulika, A., A. Perrin, et al. (1995). “Induction of homologous recombination in mammalian chromosomes by using the I-Scel system of Saccharomyces cerevisiae.” Mol Cell Biol 15(4): 1968-73.
Christian, M., T. Cermak, et al. (2010). “Targeting DNA double-strand breaks with TAL effector nucleases.” Genetics 186(2): 757-61.
Cong, L., F. A. Ran, et al. (2013). “Multiplex genome engineering using CRISPR/Cas systems.” Science 339(6121): 819-23.
Cros, E. et al. (2004). “Problems related to resistance to cytarabine in acute myeloid leukemia”. Leukemia & Lymphoma. 45(6):1123-1132.
Deltcheva, E., K. Chylinski, et al. (2011). “CRISPR RNA maturation by trans-encoded small RNA and host factor RNase III.” Nature 471(7340): 602-7.
Donnelly, M. and G. Elliott (2001). “Nuclear localization and shuttling of herpes simplex virus tegument protein VP13/14.” J Virol 75(6): 2566-74.
Doronina, V. A., C. Wu, et al. (2008). “Site-specific release of nascent chains from ribosomes at a sense codon.” Mol Cell Biol 28(13): 4227-39.
Eisenschmidt, K., T. Lanio, et al. (2005). “Developing a programmed restriction endonuclease for highly specific DNA cleavage.” Nucleic Acids Res 33(22): 7039-47.
FRANKEL S A, GERMAN W J. Glioblastoma multiforme; review of 219 cases with regard to natural history, pathology, diagnostic methods, and treatment. J Neurosurg. 1958 September; 15(5):489-503.
Gardin, C. et al. (2007). “Postremission treatment of elderly patients with acute myeloid leukemia in first complete remission after intensive induction chemotherapy:results of the multicenter randomized Acute Leukemia French Association (ALFA) 9803 trial”. Blood. 109(12):5129-5135.
Garneau, J. E., M. E. Dupuis, et al. (2010). “The CRISPR/Cas bacterial immune system cleaves bacteriophage and plasmid DNA.” Nature 468(7320): 67-71.
Gasiunas, G., R. Barrangou, et al. (2012). “Cas9-crRNA ribonucleoprotein complex mediates specific DNA cleavage for adaptive immunity in bacteria.” Proc Natl Acad Sci USA 109(39): E2579-86.
Henderson, D. J., I. Naya, et al. (1991). “Comparison of the effects of FK-506, cyclosporin A and rapamycin on IL-2 production.” Immunology 73(3): 316-21.
Jena, B., G. Dotti, et al. (2010). “Redirecting T-cell specificity by introducing a tumor-specific chimeric antigen receptor.” Blood 116(7): 1035-44.
Jinek, M., K. Chylinski, et al. (2012). “A programmable dual-RNA-guided DNA endonuclease in adaptive bacterial immunity.” Science 337(6096): 816-21.
June, C. H. et al. (2011). “T Cells with Chimeric Antigen Receptors Have Potent Antitumor Effects and Can Establish Memory in Patients with Advanced Leukemia”. Sci. Transl. Med. 3(95):ra73.
Kalish, J. M. and P. M. Glazer (2005). “Targeted genome modification via triple helix formation.” Ann N Y Acad Sci 1058: 151-61.
Li, T., S. Huang, et al. (2011). “TAL nucleases (TALNs): hybrid proteins composed of TAL effectors and Fokl DNA-cleavage domain.” Nucleic Acids Res 39(1): 359-72.
Liu, J., M. W. Albers, et al. (1992). “Inhibition of T cell signaling by immunophilin-ligand complexes correlates with loss of calcineurin phosphatase activity.” Biochemistry 31(16): 3896-901.
Lonial S, Mitsiades C. S., Richardson P. G., (2011) “Treatment options for relapsed and refractory multiple myeloma”. Clin Cancer Res. 17:1264-77.
Mali, P., L. Yang, et al. (2013). “RNA-guided human genome engineering via Cas9.” Science 339(6121): 823-6.
Morgan R A, Johnson L A, Davis J L, Zheng Z, Woolard K D, Reap E A, Feldman S A, Chinnasamy N, Kuan C T, Song H, Zhang W, Fine H A, Rosenberg S A. Recognition of glioma stem cells by genetically modified T cells targeting EGFRvIII and development of adoptive cell therapy for glioma. Hum Gene Ther. 2012 October; 23(10):1043-53. doi: 10.1089/hum.2012.041. Epub 2012 Sep. 24. Moscou, M. J. and A. J. Bogdanove (2009). “A simple cipher governs DNA recognition by TAL effectors.” Science 326(5959): 1501.
Paques, F. and P. Duchateau (2007). “Meganucleases and DNA double-strand break-induced recombination: perspectives for gene therapy.” Curr Gene Ther 7(1): 49-66.
Park, T. S., S. A. Rosenberg, et al. (2011). “Treating cancer with genetically engineered T cells.” Trends Biotechnol 29(11): 550-7.
Peipp, M., D. Saul, et al. (2004). “Efficient eukaryotic expression of fluorescent scFv fusion proteins directed against CD antigens for FACS applications.” J Immunol Methods 285(2): 265-80.
Perrin, A., M. Buckle, et al. (1993). “Asymmetrical recognition and activity of the I-Scel endonuclease on its site and on intron-exon junctions.” Embo J 12(7): 2939-47.
Pingoud, A. and G. H. Silva (2007). “Precision genome surgery.” Nat Biotechnol 25(7): 743-4.
Porteus, M. H. and D. Carroll (2005). “Gene targeting using zinc finger nucleases.” Nat Biotechnol 23(8): 967-73.
Rouet, P., F. Smih, et al. (1994). “Introduction of double-strand breaks into the genome of mouse cells by expression of a rare-cutting endonuclease.” Mol Cell Biol 14(12): 8096-106.
Sorek, R., C. M. Lawrence, et al. (2013). “CRISPR-mediated Adaptive Immune Systems in Bacteria and Archaea.” Annu Rev Biochem.
Stoddard, B. L. (2005). “Homing endonuclease structure and function.” Q Rev Biophys 38(1): 49-95.
Stupp R, Mason W P, van den Bent M J, Weller M, Fisher B, Taphoorn M J, Belanger K, Brandes A A, Marosi C, Bogdahn U, Curschmann J, Janzer R C, Ludwin S K, Gorlia T, Allgeier A, Lacombe D, Cairncross J G, Eisenhauer E, Mirimanoff R O; European Organisation for Research and Treatment of Cancer Brain Tumor and Radiotherapy Groups; National Cancer Institute of Canada Clinical Trials Group. Radiotherapy plus concomitant and adjuvant temozolomide for glioblastoma. N Engl J Med. 2005 Mar. 10; 352(10):987-96.
Van De Donk, N. W. C. J., Kamps S., Mutis, T., Lokhorst, H. M. (2012) “Monoclonal antibody-based therapy as a new treatment strategy in multiple myeloma”. Leukemia. 26:199-213.
Van Meir, E. G., Hadjipanayis, C. G., Norden, A. D., Shu, H. K., Wen, P., and Olson, J. J. (2010) “The Avenue to a Cure for Malignant Glioma” CA Cancer J Clin. 60(3): 166-193.

1. An EGFRvIII specific chimeric antigen receptor (EGFRvIII CAR) having one of the polypeptide structure selected from V1 to V6 as illustrated in FIG. 2, said structure comprising:
an extra cellular ligand binding-domain comprising a VH and a VL from a monoclonal anti-EGFRvIII antibody, optionally a linker, in particular a linker of formula (G4S)n wherein n is 1-3, preferably n=3 (of SEQ ID NO. 10),
a hinge,
a transmembrane domain and
a cytoplasmic domain including a CD3 zeta signaling domain and a co-stimulatory domain from 4-1BB.

2. An EGFRvIII specific CAR according to claim 1 comprising:
an extracellular ligand binding-domain comprising a VH and a VL from a monoclonal anti-EGFRvIII antibody, a linker, of formula (G4S)3 (of SEQ ID NO. 10),
a hinge,
a transmembrane domain from CD8 alpha and
a cytoplasmic domain including a CD3 zeta signaling domain and a co-stimulatory domain from 4-1BB.

3. An EGFRvIII specific CAR according to claim 1 comprising no domain from human CD28, in particular no co-stimulatory domain from human CD28.

4. An EGFRvIII specific CAR according to claim 1, wherein said VH and VL have at least 80% identity with a polypeptide sequence selected from SEQ ID NO. 11 to SEQ ID NO. 14, optionally humanized.

5-10. (canceled)

11. An EGFRvIII specific CAR according to claim 1, wherein said structure V1 comprises a FcγRIIIα hinge and CD8α transmembrane domain.

12. (canceled)

13. An EGFRvIII specific CAR according to claim 1, wherein said structure V3 comprises a CD8α hinge and a CD8α transmembrane domain.

14. (canceled)

15. An EGFRvIII specific CAR according to claim 1, wherein said structure V5 comprises an IgG1 hinge and a CD8α transmembrane domain.

16. (canceled)

17. An EGFRvIII specific CAR of structure V1 according to claim 1 which comprises a polypeptide sequence having at least 80% identity with SEQ ID NO. 15 or with SEQ ID NO.17.

18. An EGFRvIII specific CAR of structure V3 according to claim 1 having at least 80% identity with a sequence selected from SEQ ID NO. 24 and SEQ ID NO. 26.

19. An EGFRvIII specific CAR of structure V5 according to claim 1 having at least 80% identity with a sequence selected from SEQ ID NO. 25 and SEQ ID NO. 27.

20. (canceled)

21. A polynucleotide encoding an EGFRvIII specific CAR according to claim 1.

22. An expression vector comprising a polynucleotide of claim 21.

23. (canceled)

24. An engineered immune cell expressing at the cell surface membrane an EGFRvIII specific CAR according to claim 1.

25. An engineered immune cell according to claim 24, derived from an immune cell selected from inflammatory T-lymphocytes, cytotoxic T-lymphocytes, regulatory T-lymphocytes or helper T-lymphocytes, preferably from cytotoxic T-lymphocytes.

26. (canceled)

27. An engineered cell according to claim 24, wherein expression of TCR is suppressed.

28-29. (canceled)

30. An engineered cell according to claim 24 for use in therapy to prevent or treat a condition in a patient.

31. An engineered cell for use in therapy according to claim 30 for the treatment of a pre-malignant or malignant cancer condition characterized by EGFRvIII-expressing cancer cells.

32. (canceled)

33. An engineered cell for use in therapy according to claim 30 for use in therapy, wherein the condition is a cancer selected from lung cancer, anal cancer, residual or recurrent EGFRvIII+ Glioma, and glioblastoma multiforme (GBM).

34-35. (canceled)

36. A method of engineering an immune cell comprising:
(a) Providing an immune cell,
(b) Introducing into said cell at least one polynucleotide encoding said EGFRvIII specific CAR, according to claim 21,
(c) Expressing said polynucleotide into said cell.

37. (canceled)

38. A method of treating a subject in need thereof comprising:
(a) Providing an engineered cell according to claim 24 expressing at the surface an EGFRvIII specific CAR;
(b) Administrating said engineered cells to said patient.

39-40. (canceled)
